SEQUENCE LISTING (13 sequences)

SEQ ID NO: 1 - DNA, 11684 bp, Homo sapiens
  source 1..11684: mol_type = genomic DNA
aactcctgccaggcagtgcccttcccggagcgtgccctcgccgctggtgagtgaggacgggacgcggagggggcagcgggaagtgggggcccgcatggctgcgactcttggcagcgccgcgggacagagggaaactgaggccggagccgcagactggacacccgagggggcgacccggggcagcacttggggctcggctaccgggtcagggggcggcgggcagcagagtctgggcctccgcggccggggttccagcgcgggccgcctccggctgggggttcgggagggaaaacttggacaaccctgccacgcccagcccttggccgcgtggcttctcctgcaggaagcgcggtcccaggagtggccgacgctccctctcctgcccattccgcggatgggcaatcccaggcggaactcccttgagggtctcagaatatctgggagacctcgggctcttgatctccgagacaccccgtttcgtagtggagaacagtccagatcggggaagtttattttgcccaaagccggttagaggccccctggccctcgattcccagtgcggggctcaggtgcgttgcagcctagacgggtcttactgtgagccgagcagcctctgggacctgtctgcttcccctaccccttgcaaagaagccggagcccaggtaggagaagcgggggcttcctgctgtgaacgcgctgctggccagggcagctgccagaggccatggcctggcgtgggcctggagcccctctggccagcctgccaggggccagggctacgggataccagcagcgtgccctgggctggatggcaggagagacaggacttgaggctgtcccagaatgggctcaggcagggcgaggatatcaggggaggtggtgtacaggaagcagccgcccagcttgcctggcacacagcaagccctgcccatgaaggcctactgccagaacagtgggcgaggcccggcgtctctgtggagtcggtggggcccgggacagggcagcctgaggcaggtttccactggcggtgaaaggggccgtgtggcaaggacaggagagccagcctcagcccagcaggggaaggcggcccctgagtctccacctggctgctggcagccccacagggaggttcggcgaaactgaggcttgccaaagaagcctttgtccagagtcacgcagctggcgcggtggagccagggccagaacccgtgcaggctgatcccagcctgccttctccactgtgccccgaggaccactttctttcctgggagtgtgtgtgtgcacatgtgctgtgtcctgtcgtctgtacacacttgtgcctggcgtgaggcaggatgtctggctggatctaccagaaagtattcacagcgggttctccagctggggtgaggggcttgggctcgcagggagggagcctacttttcactttagcttgctttcttcctttcttccgtccttccttcctccctccctcccctccccttcccaccccttctttctctttcttctttctctttctttctttctttccttccttccttctttcctttctctttctctttttttctttcttccccttccttccttcctttcttgtttttgtttgtttgtttgagacagggtctcactctggtatgcaggctggagtgcagtgatgcaatcatagctcactgcagtctcctgcagaactcctgggctcaagcgatcctcccacctcagcctcccaaagtgctgggattacaggcatgagccactgttcctggcttatttgtgttttcttttttttttttttttttttttttttgagacggagtctcgctctgtcgcccaggccggactgcggactgcagtggcgcaatctcggctcactgcaagctccgcttcccgggttcacgccattctcctgcctcagcctcccgagtagctgggactacaggcgcctgccaccgcgcccggctaattttttgtatttttagtagagacggggtttcaccttgttagccaggatggtctcgatctcctgacctcatgatccacccgcctcggcctcccaaagtgctgggattacaggcgtgagccaccgcgcccggcttatttgtgttttctttacttttgatggtgcaaacaatgcttggatgtgctctctgtttaaaacggtagtcttttttatttttttttttaaccactagaccactagggaagtcattcttaatttcttatttttatttatatatatattttttgagacggtgtctcgctccattgcccaggctggagtgcagtggctccagctgggctcactgcaacctccgcctccagagttgaagtgattctcctgcctcagcctggctaatttttatatttttagtagagatgggtttcaccatgtcggccaggctgtttctttattttttactaaaaaaaagtttaacccccgaacctcgaaggaaagggaagtagtctttcttaacagaaccaattccccctggataccccaccctcaccctaccctctcccaggccactcgatggccaggttcgctgtggatttttccagacctgtgggctgtttcactggttacttttgtaatgaaaaatgcacacaaaggttacgatgaggacgattagccatgaatcatttttaaaaagcctgaaaacaagtccctgggacagtgctggcttcgttggagagacaaggctcaggggagtgggtgcagctgggggtcggggtggggatcagagaagggacacattccttattgtgtttctgggtcgccttctttttttttgtttgctttatttatttatttatttattttgagacagagtcttgctctgttgcccaggctggagtgcagtggcgtaatcttggctcactgcaacctcctcctctcgggttcaagcaattctcttgccttagccttctgagtagctgggattacagacgtgcgccaccacaccaggctagttttttatttttagtagagacggggtctcaccatgttggccaggctggtctcgaactcctgacctgaggtgatctgcctgcctcagcctcccaaagtgctgagattacaggtgtgagccaccgttcctggccttctgggtcacctatagggaggtctccaagaacagaaagacaccctgaaacccactggctcgaccaccacctcatagatggggacacagaggctcaggagatgagataccttggggcagcgatgggtctggggtcttcgggccaggagtgaagcctcacaagacctggagaccctgggtcccaccctcagtggggaagggctcagattctgcagggcccgatgctctgtgggggctgggatgttgccgcggcaggtggagagagtgatgctactggccctgagagaaccttctggcaccgttcagaggtggggtggagtggcctgcgggtccaatcctggcgccgcaggctctatgagccctgtttcagatgaggaaggaggctgagacgggcggtgacacaactcctggcaagtggtcacgcggcccagagcagcccggcagacacccagctcaaagggaggggtggggtgggttttggagtcactcggacctagaaagttctgtgacttctcatgtctcagtttcctcatctggaaaatgggaccagtcacgagtccaccatggaggccgagtgggccgtgtggctcagggagaagcagctggtcccctgtggctgctgtggggactgaacaaggcggtgaccgtcgggatgagcacggactccaccacgcaggcccctgcctaccagaggcctgtgcacaaccagagctgagcaaggcttggaaccttctcctgcctctacgtcctgcgccctctaccctgccccgatcttgcccctgttttgggggcgctgctccgtgcagggcattttcacctgtggagtgtccaggggctctcgtggctgataaggtctcgtcacaggtccaagccttgaggccctgtcttcccctggaggaaatggggctagtggccccacactgatacccacaacccctctcttctctggagttttcagggaaacgttgttgtcccaccccttcctcccttgagctaccccagccccccaagaagcagacacagcgggtttacccacctgacgttggaggaagccaggcccagagacgagccttggatggaggcagagccgaggtctggatcccgattcgctttctgaaggagacaaataaagggtgtataggtggcttcacccctctgcctgagcctgagtcctgtccctgccatgtgtgccccctggcctggcccaccccagctttccctggactcatcccagagcagatatctggatcctgcctagcctggctcagcatgactcatcatggtgggtaccgcccctgcccacctgttccccatttgggcaattcaggagctgggcagttccccagaggccctaggaaactccccgcccccgaccaggctttctccttgtggggcatctgaccgcctgttttctggtggggcagaccctctgagccccttggccctgtgggtcataaccacagccaggcctgagaagggggcagcctggggtgggagtgtgagcacgggactcagaactggcttcagaccctgctctcttgctgtggccttgggaaagccaccatgcctctgtgacttttattcgttccatacaacgggggacctggatggtggtctctaaggccctgtcagtgggaacactgggtgagtcccattgtgatttcctggggaccgagtgtgtgcctggccctggctgggtgctggagccaggtgacagaatcgtggggagttctgccacgtgctacaaggtgaggacctctagaacatggcgctcagtgaaggaagccagacacgagggccacacactgcgtgattccattgataccaaatgtccaggatgggcaagtccccagagagaagtcagattgctggcggccaggggtgggagggagggggaggggaaccactgctgatggatacggggtttcttttaggggttgaaaatgctttggaacttgatacagagggtggttgcacaacattgggaatatacaaatgctgctgaatggtgcactttaaaacggttaattttaggctgggcacggtggctcacgcctgcactgggggtttccatttgtggcatgaacatcactgaaagcgcaccagcttccatgtcgtatcaaggggttcgggatgtccacgtgtcttatgctggtggtattaaccttgctcgcctggacagggacacctgtgagtgagagcaggccattagtgtggggcaggtgggcctcaactgagggtgtcctgggaaaaggcacgtacaggcaccctggtccggctcgtttccagtggaggctgaacgaggctccttatggttcccccacaatgtcactcagctgtggactcacgcaggctccagcagagcagcggttatgtcttggttgcccagatgagcagtaggctgagctggcctgggcttacctgagaggatgtagtgggaggctgagctgggaggtggtcccagggctgtggtccagagctccatatccacttggcggtggtgcagggccagtgctcagcagtacctcctgccccagggagggtggcctttcatatgtgtagtgacatcagacccccatccccaccccatgagcacgtgtaccctggaagctggagggcggggcaggagggcggggcggggcaggagggaggggcggggcaggagggagcgggagggagaacacaggaacgtttcttgccgggacataggtcacatgcccacccccccagaatgtccatccttactgctcatttttgcagcgaccttgggggatggggtaactgtccccattcaacaggcgagaaaactgaggtccagggaggggagggaatcatagtaagtggcagcaccggaatctgctggatgcctgacactctgttcctttaaaacgtgccgggcgcggtggctcacgcctgtaatcccagcactttgggaggctgaggcaggcagatcaactcaggtcaggagtttgagaccagcccggccaacatggtgaaaccctgtctctactaacaatacaaaaattagcccggcatggtggcacatgctggtaatcccagctacttgggaggcaggaggctgagacgggagaatcccctgaacctgggaggcagaggttacagtgagctgagatcgcgccattgtattccaccctgggcaacagagcaagactctgtctcaaaaaaagcgcatatctatgttatccattttttgccaaaattggggtcattctgtagtcaatgttatattctgctttcttcgtgttatgttgagaacaactttctcgttactagaaagccttcagaagcttggtttgtggggggccagtctttaatagtccctggctgctgggcactgaggttgcttctgtgctcacccagctccatggatcccaggccagcagccgccccttctccctggggccagatttgccctttgccctttgcccctgccaaagcccaagttcagcacagtctctgctgacacccctctacccccgcttggctgcctccatgtcccctggactgcttgtctctcgtgttcccctgggacgaaagtgctcagtattcttcctgtggcatctcctccctggtccctggtcagagggtccctttctgaggtccaggctccctcagcaccagtctgtgcccactcagggcctccccagccctgtgcccccctgggtcctagccctcgccccagcaggccttgctgcaagttggcagaactgaatcgggttgtgtggcttcttgtctcagacaagagcagacgactcctgattctccacctagaatcagagtgagtgggagcttagccctttgtttttggatgaggtcagggtgggagccatattgccaccagggcagagcctgggccagagtgcagtgtggccccaggcaggctgctctctgtctccggtgcatggggaaggggcggtggttttactggggcccgtgtggggtgctgaaacagctgtgtagagaccttccttgctgctgtgcactgagctgtgctcaaggtaggtgagcattgatatcccactctaccgagggggaaactgaggccctgagttgatgtgatttgcctgaggtcacagtgaggcagtgaggaaggggattagacccaaactctgtctgataccagtcaccaaatgaatgagtgagtgagtgagtgagtgaaaggtgaatgtgaagtgctggaacgtaaagcactggcctggcagagctcccaggacggggacttgacccagggaggcaggctgggaaggcttcttggtggaggggacccaaactggccttggaagagaggaggggtcagtgccagagacccaaggtcgaggtgagggtgggcaaaggtgaggcgaggcagctggggcaggagcaaggctggaaatgcgttcactcagccactcccaccccgcacatctctgcgcctcctgatcagctgactggttgatctgtgggtgacaggcctttcggggatgctggaggagacacgggcctgaccccatggcctcaggaatgccctcacccagccctgtcttctctttcagagctcccctgaacagcagctgcagcagccatggccccgccctgggtgcccgccatgggcttcacgctggcgcccagcctggggtgcttcgtgggctcccgctttgtccacggcgagggtctccgctggtacgccggcctgcagaagccctcgtggcacccgccccactgggtgctgggccctgtctggggcacgctctactcagccatggggtaggtgggcgtgcactggcctggggataagcctggccctttgcaaggggaggcctggcccaggacagagggtcttctccaggcgggccatggaccatggcatggtttccccagccccatttggcaagccagggtggggaagctgtgggcctgtcgattgcacaactgaactttctcctcctgggcccctggggggatgggtcagatgctcaccccaccctggcatccccgccatgttcacagctccaggcagaggccggcttagtgtgtgaaggttggtcttgggcagaggcatggacgctaagattaacaacggcagcaacagccccaagagcagctcctttgttgaacgcctgcccagtgccaggctcgtctcattcatactcaacgacccccataataagaggcctcccaaggacacagggcttgcaagtgtggaccccccacagccccttcaggctcagtctggcctgagtcccctgggaatagaccaggggaattccacccggaaaggacgtgctgggaggggcagggcgcctcttggtggctgtagcggggaccacacccaggaggcagggaattgactgccttctgaaatgttccctgcatcctggttctgtgccagcgcagtgcagggtggggacgcaggtgccaggcactctagggggcggatagcagaggccaggccaggtgcccggctctgatgggaataaggccctggaacttgggtgtcacgtgggcctcacccctcctggcccaggccctgcccagccagggtcctgcacttccctggctccagtgtccccaccctgcttggccttcctacaccacccagtcccagcctctcttcgggggaaattttggggctccctgtggctctggataaggcgaacccctcattagtaggtgacagccggggctctggccctggctgaccagactttatccactgatggcctgccaagagatctcagtcacttcaccgcctcagtctcctcactgtaaagggggtgctgatactgcctgccttatggcatcatcatgagactgaaatgagtgaatgcttgggaagtggtagggcctggctcctgtgagtgctggagggcatcggctgctgttactggtggtgttatcgttgccctgtgttaacagctcccatgatcaggcctccccctggacctcaccctgcagtcacagactgactcagggtctcacatctgcctcaaagcctttgcatgtcctgtttcctcccctggaaaactcctattcatcatttaaggcccaggccaaatgtcccatcctctatgcagtgccctggctaggttaagagcctcctctctacctattcacaccatgtgcttccctttcatagcagtgacagtgataataatggcaggtatttactgagtgtcctaaatgctgggtatcgcatgaagcactgccaatgtgctaactagaatctctacagcaaccctatgaaaaatgaaggcccagacaggtcaagcaacttccctgagatcacacagcagtcagtgtcaggtcacgtatgaaccatcactgtgccactcggaggtgggcaacgctcctggccttgttcctaatggtgctctgaactgcggcctctgtttcaggtacggctcctacctggtctggaaagagctgggaggcttcacagagaaggctgtggttcccctgggcctctacactgggcagctggccctgaactgggcatggccccccatcttctttggtgcccgacaaatgggctgggtaagtgtggccacagcatgtgtccctgatccctggatccgacccttggaggacgtggggcatcacatatgacactgggtcagtgtctataggcggggccaggggagacaaaaggccatgtctctctagctgtaagcagcccacaccctggagcctcccctctactgactcctccggtgagggaagctattaaagcagaaggggttgcaggggtgggtttgggggccactgtgtaggaaaacccacacgaagcctggtactgtgtgggcagggtcactggcccctctacataacagcttcttcttttttttttttgagacagagtctcactctctcacccaggctggagtgcagtggtgagatctcggcccactgcaacctccacctcctgggttcaagtgattcccttgcctcagcctcccgagtagctgggactacaggtgtgcaccaccactcctggctaatttttgtatttgtatttgtatttttttagtagagatggggtttcactatgttggccaggctggtcttgaactcctgacctcaggtgatctacccgccttggcctctcaaagtgctgggattacaggtgtaagccaccgcgcccggctgactgcttcttttttaaagttttaaacttttttaagagatgaagactcgctgtgttgcccagggcagactcgacttcctgggctcaaacgattgattctcccgcctttacctcttgagtagctgggactccaggtcacgtcactgtgcccggcagccttttttttttgagacagagtcttactctgttgcccaggctggagtgcagtggcatgatctcggctcactgcagactccacctcccaggttcaagtgattctcctgtctcagcctcccgagtagctgggattacaggcatgcagcaccatgtccagctaattttctgtatttttttttttttgagacggagtctcgctctgttgcccaggctggagtgcagtggcgtgatctcagctcactgcaacctccgtctcccaggttcaagcaattctcctgcctcagcctcctgagtagctgggactacaggcacgtgctaccacgcctggctaatttttttatttttagtagagacggggttttaccatattggtcaggttggtcttgaactcctgacctcaggtgatccacccgtctcagcctcccaaagtgctgggattacaggcgtgagccactgcacctgacaaatagcaagtttttgtttgggccttgtagtaatgctgggaggtcagcatcgctctcagtggaggctctaagctcagagaggggaagggacgtgcaagacctcctaagccaccaccgcacccttgaacaaaccgagttcacttcaggcatgtcatgtgcaccccacacaaggcatgggtcaggtggcatgactgttcccattttacagatgaggaaactgaggctgcgatgggggaggggcttggccaggtcactcaagggtggagtgggggtgagtgaggctcctgactcccaaatccagtgggagttgggcagtgggacaggcacttgggtgaacgcggtgcctcaggcctccccatcctccgtcccccaatctctgcaggccttggtggatctcctgctggtcagtggggcggcggcagccactaccgtggcctggtaccaggtgagcccgctggccgcccgcctgctctacccctacctggcctggctggccttcacgaccacactcaactactgcgtatggcgggacaaccatggctggcgtgggggacggcggctgccagagtgagtgcccggcccaccagggactgcagctgcaccagcaggtgccatcacgcttgtgatgtggtggccgtcacgctttcatgaccactgggcctgctagtctgtcagggccttggcccaggggtcagcagagcttcagaggtggccccacctgagcccccacccgggagcagtgtcctgtgctttctgcatgcttagagcatgttcttggaacatggaattttataagctgaataaagtttttgacttccttta

SEQ ID NO: 2 - protein, 169 aa, Homo sapiens
MAPPWVPAMGFTLAPSLGCFVGSRFVHGEGLRWYAGLQKPSWHPPHWVLGPVWGTLYSAMGYGSYLVWKELGGFTEKAVVPLGLYTGQLALNWAWPPIFFGARQMGWALVDLLLVSGAAAATTVAWYQVSPLAARLLYPYLAWLAFTTTLNYCVWRDNHGWRGGRRLPE

SEQ ID NO: 3 - protein, 121 aa, Severe acute respiratory syndrome coronavirus 2
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 4 - DNA, 366 bp, Severe acute respiratory syndrome coronavirus 2
  source 1..366: mol_type = genomic DNA
atgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaa

SEQ ID NO: 5 - protein, 275 aa, Severe acute respiratory syndrome coronavirus 2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 6 - DNA, 828 bp, Severe acute respiratory syndrome coronavirus 2
  source 1..828: mol_type = genomic DNA
atggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaa

SEQ ID NO: 7 - RNA, 21 nt, synthetic construct
  source 1..21: mol_type = other RNA
cccatcttctttggtgcccga

SEQ ID NO: 8 - RNA, 21 nt, synthetic construct
  source 1..21: mol_type = other RNA
accattgggcctgctggtcta

SEQ ID NO: 9 - RNA, 21 nt, synthetic construct
  source 1..21: mol_type = other RNA
tcctggtcgctgaaccttcca

SEQ ID NO: 10 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 11 - DNA, 1260 bp, Severe acute respiratory syndrome coronavirus 2
  source 1..1260: mol_type = genomic DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 12 - protein, 75 aa, Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV

SEQ ID NO: 13 - DNA, 228 bp, Severe acute respiratory syndrome coronavirus 2
  source 1..228: mol_type = genomic DNA
atgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

(19) United States
(12) Patent Application Publication
(10) Pub. No.: US 2025/0019701 A1
(43) Pub. Date: Jan. 16, 2025

(54) TREATMENT FOR INFLAMMATORY DISEASE

(71) Applicant: THE ROYAL VETERINARY COLLEGE, London (GB)

(72) Inventors: Michelangelo CAMPANELLA, London (GB); Aarti SINGH, London (GB)

(21) Appl. No.: 18/707,964

(22) PCT Filed: Nov. 8, 2022
(86) PCT No.: PCT/GB2022/052816

§ 371 Date: May 7, 2024

(30) Foreign Application Priority Data
Nov. 9, 2021 (GB) 2116071.8

(51) Int. Cl. C12N 15/113; A61K 31/4045; A61K 31/472; A61K 31/5025; A61K 31/522; A61K 31/536; A61P 29/00; C12N 9/22; C12N 15/86; G01N 33/50; G01N 33/68

(52) U.S. Cl. CPC C12N 15/113; A61K 31/4045; A61K 31/472; A61K 31/5025; A61K 31/522; A61K 31/536; A61P 29/00; C12N 9/22; C12N 15/86; G01N 33/5047; G01N 33/6893; C12N 2310/11; C12N 2310/141; C12N 2310/20; G01N 2800/7095

(57) ABSTRACT

The present disclosure relates to a method of treating or preventing a disease in an individual, the method comprising reducing pyroptosis by administering to the individual a composition comprising an agent whose administration reduces TSPO signalling in the individual. The method of the disclosure can thus be used to treat or prevent diseases associated with pyroptosis such as inflammatory disease, cancer, neurodegenerative disease, cardiovascular disease, kidney disease and sepsis. The disclosure also relates to use of TSPO expression as a biomarker for pyroptosis, a method of assessing the degree of pyroptosis in an individual based on TSPO expression, an in vitro model of pyroptosis, a method of producing the in vitro model and associated vector, and a method for determining the ability of an agent to inhibit pyroptosis.

### FIELD OF THE DISCLOSURE

The present disclosure relates to a method of treating or preventing a disease in an individual, the method comprising reducing pyroptosis by administering to the individual a composition comprising an agent whose administration reduces TSPO signalling in the individual. The method of the disclosure can thus be used to treat or prevent diseases associated with pyroptosis such as inflammatory disease, cancer, neurodegenerative disease, cardiovascular disease, kidney disease and sepsis. The disclosure also relates to use of TSPO expression as a biomarker for pyroptosis, a method of assessing the degree of pyroptosis in an individual based on TSPO expression, an in vitro model of pyroptosis, a method of producing the in vitro model and associated vector, and a method for determining the ability of an agent to inhibit pyroptosis.

### BACKGROUND

Pyroptosis is a highly inflammatory form of lytic programmed cell death that occurs upon infection with intracellular pathogens. The pathogenesis of many other diseases, such as cancers, neurodegeneration, cardiovascular conditions and kidney conditions also involve pyroptosis.

In certain circumstances, pyroptosis may be beneficial as it is involved in the defensive response mechanisms against intracellular pathogens. Pyroptosis operates to remove the replication niche of intracellular pathogens, making them susceptible to phagocytosis and killing by a secondary phagocyte. Many pathogens have, though, evolved to exploit pyroptosis, including coronaviruses such as SARS-CoV-2. Furthermore, pyroptosis is an acknowledged exacerbating factor in many diseases. Aberrant and systemic activation of pyroptosis in vivo may contribute to sepsis, and/or damage vital organs. Many debilitating manifestations of various diseases are associated with inflammation caused by a high level of pyroptosis. For instance, pyroptosis in COVID-19 patients damages lung epithelium and cardiovascular endothelium, and compromises the innate immune response.

Currently, the agreed general strategy to deal with the SARS-CoV-2 pandemic is based on preventive treatments and anti-viral therapeutics. To date there are few effective therapies to assist those who are not vaccinated against SARS-CoV-2 or for who experience breakthrough infection. Furthermore, it is clear that new SARS-CoV-2 variants arise frequently, and it is possible that existing vaccinations may not work so well against such variants. Further, effective treatments for SARS-CoV-2 are therefore required. In addition, there is an ongoing need to seek therapeutic alternatives for diseases which bear an inflammatory component, such as pyroptosis-associated diseases.

### SUMMARY OF THE DISCLOSURE

In addressing the need for treatments of diseases involving pyroptosis, the inventors have identified a role for TSPO (the 18 kDa translocator protein) in promoting this type of cellular demise which occurs as a consequence if engaging the inflammasome. The inventors have shown for the first time that pyroptosis may be reduced using an agent that reduces TSPO signalling e.g., by reducing activity or by repression of the protein itself. The inventors have therefore identified that such an agent may be used to treat diseases associated with inflammation, such as COVID-19. Identification of TSPO's role in promoting pyroptosis has also enabled the inventors to envisage the use of TSPO as a biomarker for pyroptosis. In addition, they have demonstrated that inducing signalling downstream of TSPO allows for in vitro modelling of pyroptosis. This in vitro model may be used to screen for further agents that may be used to inhibit pyroptosis.

The disclosure therefore provides a method of treating or preventing a disease in an individual, the method comprising reducing pyroptosis by administering to the individual a composition comprising an agent whose administration reduces TSPO signalling in the individual. The disclosure also provides:
a composition for use in a method of treating or preventing a disease in an individual, the method comprising reducing pyroptosis by administering the composition to the individual, and the composition comprising an agent whose administration reduces TSPO signalling in the individual;
use of TSPO expression as a biomarker for pyroptosis;
a method of assessing the degree of pyroptosis in an individual, comprising measuring the amount of TSPO expressed in a sample obtained from the individual, wherein the amount of TSPO expressed in the sample correlates with the degree of pyroptosis in the individual;
an in vitro model of pyroptosis, comprising cells modified to comprise a polynucleotide encoding one or more virus-related proteins that activate the NLRP3 inflammasome;
a vector comprising a polynucleotide encoding one or more virus-related proteins that activate the NLRP3 inflammasome;
a method of producing an in vitro model of pyroptosis, comprising introducing the vector of the disclosure to cells and expressing the one or more virus-related proteins in the cells; and
a method for determining the ability of an agent to inhibit pyroptosis comprising: (a) culturing a first population of cells and a second population of cells for a period of time, wherein (i) the first population and the second population are obtained from the in vitro model of the disclosure, and (ii) the first population is cultured in the presence of the agent and the second population is cultured in the absence of the agent; (b) determining the level of pyroptosis in each of the first and second populations; and (c) using the level determined in (b) to indicate the ability of the agent to inhibit pyroptosis, wherein reduced pyroptosis in the first population relative to the second populations indicates that the agent is capable of inhibiting pyroptosis.

### BRIEF DESCRIPTION OF THE FIGURES

FIG. 1 shows that TSPO is required for pyroptotic cell death in murine immune cells of the brain (BV-2) (ABC-TC212S). A shows a western blot showing TSPO protein levels in WT BV2 cells and in selected negative (C1), heterozygous (C2) and homozygous (C3) TSPO KO (knockout) clones. The blot confirms effective CRISPR/Cas9 gene editing-mediated ablation of TSPO in clone C3. B. shows quantification of TSPO protein levels in control and TSPO KO clones based on densitometry analyses carried out on the western blot of A. C shows the growth curve of WT and TSPO KO BV2 cells showing a significantly lower doubling time (higher proliferation) in the latter. D shows quantification of changes in cell death as measured by PI (propidium iodide) inclusion between WT and KO cells treated with LPS (lipopolysaccharide) (24 hours using 100 ng/mL) or LPS+ATP (24 hours using 100 ng/mL followed by 30 min using 2.5 mM ATP) to induce pyroptosis. TSPO KO thus protects from LPS-induced pyroptosis. E shows western blotting analysis of NLRP3 protein levels in WT and TSPO KO BV2 cells at rest (controls are vehicle and carbonyl cyanide-p-trifluoromethoxyphenylhydrazone also referred to as FCCP) and after LPS- or LPS/ATP-induced activation (as described above). F shows the changes in NLRP3 band density relative to the band density of ACTB (Beta-actin) from the representative western blot of E, indicating that both treatments induce a significant upregulation in NLRP3 protein expression in WT cells only, while NLRP3 levels do not change when TSPO is ablated, as quantified in F. G shows a representative immunoblotting analysis of NF-κB in the nucleus following treatment with vehicle, FCCP, LPS and LPS+ATP. H shows respective densitometry analyses indicating that treatment with LPS or LPS/ATP causes a significant translocation of NF-κB into the nucleus in WT cells, which is hampered by the lack of TSPO.

FIG. 2 shows that SARS-CoV-2 proteins ORF8.2b and ORF3a stabilise TSPO. A shows an immunoblotting analysis of the NRLP3 expression level following THP-1 cells transfection with ORF8.2b and ORF 3a. B shows the relative quantification of the band densities from the immunoblot of A. C shows an immunoblotting analysis of the TSPO expression level following THP-1 cells transfection with ORF8.2 and ORF 3a. D shows the relative quantification of the band densities from the immunoblot of C. E shows an immunoblotting analysis of the NRLP3 expression level following A549 cells transfection with ORF 8.2b and ORF 3a. F shows the relative quantification of the band densities from the immunoblot of E. G shows an immunoblotting analysis of the TSPO expression level following A549 cells transfection with ORF 8.2b and ORF 3a. H shows the relative quantification of the band densities from the immunoblot of G.

FIG. 3 shows that TSPO ligands confer cytoprotection to cells expressing the SARS-CoV-2 encoded proteins ORF 8.2b and ORF 3a which trigger pyroptosis. A shows a scheme of THP-1 cells transfected with ORF 8.2b and ORF 3a on which treatment with TSPO ligands was then performed. B-E show an assessment of cell death by monitoring the release of lactate dehydrogenase (LDH) indicative of alterations in membrane permeability of THP-1 cells expressing SARS-CoV-2 derived proteins ORF 8.2b and ORF 3a and treated with TSPO ligands, etifoxine, PK11195, XBD173 and FGIN respectively. F-I show an assessment of cell death by monitoring the release of lactate dehydrogenase (LDH) indicative of alterations in membrane permeability of A549 cells expressing SARS-CoV-2 derived proteins ORF 8.2b and ORF 3a and treated with TSPO ligands, etifoxine, PK11195, XBD173 and FGIN respectively.

FIG. 4 shows that SARS-CoV-2 infection upregulates TSPO. A shows a diagram referring to the protocol adopted to infect Vero cell with SARS-CoV-2 with the varying multiplicity of infection (MOI) ratios specular to the proportion of viral particles. Following either 24, 48 and 72 hours post infection cells were harvested and protein content isolated for examination through western blotting. B shows the expression of Caspase 8 (Casp 8-FL depicting full-length caspase-8 and Casp 8-CL depicting caspase-8 that is cleaved) and TSPO as well as house-keeping gene HSP90 essential to normalise protein levels and run the quantification presented in C. As highlighted in the boxes, when MOI increases from 0 to 10000 virions there is a large increase, statistically significant, in TSPO expression. C shows quantification of the ratio of TSPO to HSP90 based on the western blot of B.

FIG. 5 shows that TSPO ligands protect from SARS-CoV-2 triggered pyroptosis. A shows the proliferation of Vero cells 24 hours and 48 hours post-infection using SARS-CoV-2 virions in untreated conditions or following treatment with TSPO ligands, etifoxine (30 μM), FGIN 1-27 (200 nM), PK11195 (200 nM) and XBD 173 (30 μM). The tangible visual indication of the protection conferred by ligand treatment is shown. B is a quantitative analysis based on counting adherent cells which further confirms and statistically proves the protection by TSPO ligands shown in A. This data shows that infection alone causes a large degree of cell death after 48 hour of infection which is counteracted by the TSPO ligands. Among the chemicals enrolled in the analysis, etifoxine reduced cell death by approximately 50% (statistically significant) which is highly indicative of its potential as a pharmacological inhibitor of COVID-19 associated cell death.

FIG. 6 shows representative images from a propidium iodide (PI) assay performed in THP-1 cells to assess transfection efficiency and cell death following transfection using either plasmid containing ORF8 from SARS-CoV (and a fluorophore) or GFP. Cell death was assessed at 24, 48 and 72 hours post transfection. 6b shows the quantification of the percentage cell death quantified and a higher level of cell death is measured following transfection especially with the ORF8 protein. The data show that 24 hours of transfection with a plasmid containing ORF8 from SARS-CoV is sufficient to see a substantial increase in cell death compared to following transfection of a vector containing a fluorophore alone

FIG. 7 relates to analysis of cell death using caspase expression. 7a shows shows representative blots of Caspase and ASC protein normalized to house-keeping gene Vinculin in THP-1 cells. 7b and 7c show the quantification of Caspase 1 and ASC levels assessed in THP-1 cells following transfection of SARS-CoV-2 proteins. FIGS. 7d and 7e show representative blots of ASC in A549 following transfection using either low or high concentrations of ORF3 or 8 of SARS-CoV-2, where there is only an increased quantification following administration of low levels of ORF3a plasmid.

FIG. 8 concerns cell death in THP-1 cells and A549 cells treated with TSPO ligands. FIG. 8a-d shows quantification of PI cell death in THP-1 cells at 24 hours and 48 hours after ligand treatment. FIG. 8e-h shows quantification of PI cell death in A549 at 24 hours and 48 hours after ligand treatment, with a significant decrease observed at 48 hours after ORF3a transfection and treatment with Etifoxine, FGIN, PK and XBD.

FIG. 9 concerns a co-immunoprecipitation study in THP-1 cells to assess whether there is an indirect or direct interaction between TSPO and the proteins that make the NLRP3 inflammasome. FIG. 9a shows a representative a representative blot of ASC and TSPO co-immunoprecipitation and the loading control beta actin, before and after transfection of SARS-CoV-2 ORF proteins. FIG. 9b shows a representative blot of ASC and TSPO co-immunoprecipitation and the loading control vinculin, before and after transfection of SARS-CoV-2 ORF proteins and then followed by Etifoxine or PK11195 treatment.

FIG. 10 is an updated version of FIG. 2. Like FIG. 2, FIG. 10 shows that SARS-CoV-2 proteins ORF8.2b and ORF3a stabilise TSPO. A shows an immunoblotting analysis of the NRLP3 expression level following THP-1 cells transfection with ORF8.2b and ORF 3a. B shows the relative quantification of the band densities from the immunoblot of A. C shows an immunoblotting analysis of the TSPO expression level following THP-1 cells transfection with ORF8.2 and ORF 3a. D shows the relative quantification of the band densities from the immunoblot of C. E shows an immunoblotting analysis of the NRLP3 expression level following A549 cells transfection with ORF 8.2b and ORF 3a. F shows the relative quantification of the band densities from the immunoblot of E. G shows an immunoblotting analysis of the TSPO expression level following A549 cells transfection with ORF 8.2b and ORF 3a. H shows the relative quantification of the band densities from the immunoblot of G.

### DESCRIPTION OF THE SEQUENCE LISTING

SEQ ID NO: 1—DNA sequence of the TSPO gene (>NC_000022.11:43151559-43163242 Homo sapiens chromosome 22, GRCh38.p13 Primary Assembly)

SEQ ID NO: 2—amino acid sequence of the TSPO protein. (>spP30536 TSPO_HUMAN Translocator protein OS=Homo sapiens OX=9606 GN=TSPO PE=1 SV=3)

SEQ ID NO: 3—amino acid sequence of ORF 8.2b SARS-CoV-2 (>YP_009724396.1 ORF8 protein-severe acute respiratory syndrome coronavirus 2)

SEQ ID NO: 4—polynucleotide sequence encoding ORF 8.2b SARS-CoV-2 (>NC_045512.2:27894-28259-severe acute respiratory syndrome coronavirus 2 isolate Wuhan-Hu-1, complete genome).

SEQ ID NO: 5—amino acid sequence of ORF 3a SARS-CoV-2 (>YP_009724391.1 ORF3a protein-severe acute respiratory syndrome coronavirus 2)

SEQ ID NO: 6—polynucleotide sequence encoding ORF 3a SARS-CoV-2 (>NC_045512.2:25393-26220 Severe acute respiratory syndrome coronavirus 2 isolate Wuhan-Hu-1, complete genome).

SEQ ID NO: 7—siRNA sequence targeting human TSPO.

SEQ ID NO: 8—siRNA sequence targeting murine TSPO.

SEQ ID NO: 9—siRNA sequence targeting canine TSPO.

SEQ ID NO: 10—amino acid sequence of SARS-CoV-2 nucleocapsid (N) protein (YP_009724397.2).

SEQ ID NO: 11—polynucleotide sequence encoding SARS-CoV-2 nucleocapsid (N) protein (Gene ID: 43740575).

SEQ ID NO: 12—amino acid sequence of SARS-CoV-2 Envelope (E) protein (YP_009724392.1).

SEQ ID NO: 13—polynucleotide sequence encoding SARS-CoV-2 Envelope (E) protein (Gene ID: 43740570).

### DETAILED DESCRIPTION

It is to be understood that different applications of the disclosed methods, models, and products may be tailored to the specific needs in the art. It is also to be understood that the terminology used herein is for the purpose of describing particular embodiments of the disclosure only, and is not intended to be limiting.

All publications, patents and patent applications cited herein, whether supra or infra, are hereby incorporated by reference in their entirety.

### General Definitions

Unless defined otherwise, technical and scientific terms used herein have the same meaning as commonly understood by a person skilled in the art to which this disclosure belongs.

As used in this specification and the appended claims, the singular forms “a”, “an”, and “the” include plural referents unless the content clearly dictates otherwise. Thus, for example, reference to “a cell” includes “cells”, reference to “an antisense oligonucleotide” includes two or more such antisense oligonucleotides, and the like.

In general, the term “comprising” is intended to mean including but not limited to. For example, the phrase “a composition comprising an agent” should be interpreted to mean that the composition contains an agent, but that the composition molecule may contain additional components such as other agents.

In some aspects of the disclosure, the word “comprising” is replaced with the phrase “consisting of”. The term “consisting of” is intended to be limiting. For example, the phrase “a composition consisting of an agent” should be interpreted to mean that the composition contains an agent and no additional components.

The terms “protein” and “polypeptide” are used interchangeably herein, and are intended to refer to a polymeric chain of amino acids of any length.

For the purpose of this disclosure, in order to determine the percent identity of two sequences (such as two polynucleotide sequences), the sequences are aligned for optimal comparison purposes (e.g., gaps can be introduced in a first sequence for optimal alignment with a second sequence). The nucleotide residues at nucleotide positions are then compared. When a position in the first sequence is occupied by the same nucleotide residue as the corresponding position in the second sequence, then the nucleotides are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence has a certain percentage identity to SEQ ID NO: X. SEQ ID NO: X would be the reference sequence. For example, to assess whether a sequence is at least 80% identical to SEQ ID NO: X (an example of a reference sequence), the skilled person would carry out an alignment over the length of SEQ ID NO: X, and identify how many positions in the test sequence were identical to those of SEQ ID NO: X. If at least 80% of the positions are identical, the test sequence is at least 80% identical to SEQ ID NO: X. If the sequence is shorter than SEQ ID NO: X, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm

### Method of Treatment and Medical Use

The disclosure provides a method of treating or preventing a disease in an individual, the method comprising reducing pyroptosis by administering to the individual a composition comprising an agent whose administration reduces TSPO signalling in the individual. The disclosure also provides a composition for use in a method of treating or preventing a disease in an individual, the method comprising reducing pyroptosis by administering the composition to the individual, and the composition comprising an agent whose administration reduces TSPO signalling in the individual.

As explained above and below, pyroptosis is a form of inflammatory cell death that is involved in the pathogenesis of many diseases. Reduction of pyroptosis (and thus inflammation) may improve the clinical signs and/or outcome of the diseases. Presently, though, few anti-pyroptotic agents are known.

As shown in the Examples, the inventors have established for the first time that TSPO is required for pyroptosis. In particular, TSPO commits mitochondria to inflammatory response and driven cell death. The inventors have further shown that agents that reduce TSPO signalling can be used to reduce pyroptosis, i.e. as anti-pyroptotic drugs. The inventors have also shown that proteins derived from viruses (such as SARS-CoV-2) exploit TSPO to induce pyroptotic cell death. Agents that reduce TSPO signalling (including TSPO ligands and nucleic acid silencing molecules) can therefore be used to treat viral diseases such as COVID-19.

Agents that reduce TSPO signalling are already known in the art. For example, TSPO ligands have previously been used to score and reduce brain inflammation, and to treat neurological disorders. For instance, etifoxine is used an axioloytic, and PK11195 has been used for diagnostic imaging of brain injury. The present inventors are the first to identify that these known agents may be repurposed to reduced pyroptosis and therefore treat many other diseases. This is a particularly important finding, because pyroptosis is currently devoid of process-specific regulators capable of conferring cytoprotection.

### Pyroptosis

Pyroptosis is a form of programmed cell death that is involved in many disease processes. Pyroptosis is triggered by pro-inflammatory signals, and is associated with inflammation. Pyroptosis may therefore be considered to be a form of inflammatory cell death.

Pyroptosis relies on the activation of caspase-1. Caspase-1 is activated during pyroptosis by a large supramolecular complex known as the inflammasome. Caspase-1 activation mediates the maturation and secretion of pro-inflammatory cytokines, such as IL-1ß and IL-18. Caspase-1-dependent pore formation leads to rupture of the plasma membrane of the pyroptotic cell, and the pro-inflammatory cytokines are released. Pyroptosis is also associated with DNA cleavage and nuclear condensation.

In the method of treatment and medical use of the disclosure, administration of the composition reduces pyroptosis. Reduction of pyroptosis in turn treats or prevents the disease.

Administration of the composition may, for example, reduce pyroptosis in the individual. For instance, administration of the composition may reduce pyroptosis relative to the level of pyroptosis in the individual prior to administration. Methods for determining the level of pyroptosis in an individual are known in the art. For example, serum biochemistry may be used to determine the level of pyroptosis. Indicators of pyroptosis may, for example, include ASC (the adaptor molecule apoptosis-associated speck-like protein containing a CARD), caspase-1, IL-1B, IL-18, GSDMD and HMGB1 (High mobility group box protein 1). Changes in the level of one or more of these proteins in a sample (e.g. a blood or serum sample) obtained from the individual may indicate a corresponding change in the level of pyroptosis. For example, reduction in one or more of ASC, caspase-1, IL-1B, IL-18, GSDMD and HMGB1 following administration of the composition may indicate reduced pyroptosis in the individual compared to before administration.

### Disease

As set out above, administration of the composition reduces pyroptosis. Reduction of pyroptosis in turn treats or prevents the disease in the individual.

Accordingly, the disease that is treated or prevented in the individual may be any disease that is characterized by pyroptosis. The disease may, for example, be a pyroptosis-associated disease. A disease may be characterised by pyroptosis, or considered a pyroptosis-associated disease, if pyroptosis is involved in its pathogenesis. For instance, pyroptosis may contribute to the clinical signs of the disease. Pyroptosis may contribute to progression of the disease.

As set out above, pyroptosis is induced by and causes inflammation. Reduction of pyroptosis by administering the composition may, therefore, reduce or prevent inflammation. Thus, the method of treating or preventing a disease in an individual may comprise reducing inflammation by administering the composition. The method of treating or preventing a disease in an individual may be a method of treating or preventing inflammation in the individual.

The disease may be any disease that is characterised by inflammation. In other words, the disease maybe an inflammation-associated disease. A disease may be characterised by inflammation, or considered an inflammation-associated disease, if inflammation is involved in its pathogenesis. For instance, inflammation may contribute to the clinical signs of the disease. Inflammation may contribute to progression of the disease.

Inflammation is involved in the pathogenesis of many diseases. For example, inflammation may be involved in the pathogenesis of cancer. The disease may, therefore be a cancer. The cancer may be a solid tumour, For example, the cancer may be non-small cell lung cancer, squamous cell carcinoma of head and neck (SCCH), squamous cell carcinoma (e.g. laryngeal squamous cell carcinoma), pancreatic cancer or glioma. Pyroptosis (and therefore inflammation) has been shown to be associated with all of these cancers (Gao et al., 2018 Oncol Rep; Huang et al., 2017 J Exp Clin Cancer Res, Hue et al., 2019 Front Oncol, Boone et al., 2019 Ann Hematol, Yin et al., 2018 Int J Oncology).

Inflammation may, for example, be involved in the pathogenesis of liver disease, cardiovascular disease, neurological disease, or kidney disease. Therefore the disease may be liver disease, cardiovascular disease, neurological disease, or kidney disease. The liver disease may, for example, be hepatitis such as hepatitis C. Pyroptosis (and therefore inflammation) has been shown to be associated with hepatitis C (Kohafi et al., 2016 Sci Rep). The cardiovascular disease may, for example, be myocardial infarction. Pyroptosis (and therefore inflammation) has been shown to be associated with myocardial infarction (Mezzaroma et al, 2011 PNAS). The neurological disease may, for example, be a neurodegenerative disease such as Alzheimer's disease, Parkinson's disease, multiple sclerosis or amyotrophic lateral sclerosis. The disease may be sepsis, which involves widespread inflammation.

The disease may be a so-called inflammatory disease. In the context of the disclosure, an inflammatory disease may be defined as a disease or disorder that is caused by an inflammatory abnormality. Many inflammatory diseases are well known in the art. The inflammatory disease may, for example, be allergy, asthma, autoimmune disease, coeliac disease, glomerulonephritis, hepatitis, inflammatory bowel disease, reperfusion injury, graft-versus-host disease (GvHD) or transplant rejection. The allergy may, for example, be atopic dermatitis, allergic airway inflammation or perennial allergic rhinitis. The autoimmune disease may be, for example, alopecia areata, autoimmune encephalomyelitis, autoimmune hemolytic anemia, autoimmune hepatitis, dermatomyositis, diabetes (type 1), autoimmune juvenile idiopathic arthritis, glomerulonephritis, Graves' disease, Guillain-Barré syndrome, idiopathic thrombocytopenia purpura, myasthenia gravis, autoimmune myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, autoimmune thyroiditis, uveitis or vitiligo.

Inflammation may be involved in the pathogenesis of infectious diseases. Therefore, the disease may be an infectious disease. The infectious disease may, for example, be a viral disease, a bacterial disease, a fungal disease or a protozoal disease. Viral diseases may include diseases caused by any of the viruses disclosed herein. The viral disease may, for example, be a coronaviral disease. That is, the viral disease may be infection with a coronavirus. The coronavirus may, for example, be severe acute respiratory syndrome coronavirus 2 (SARS-CoV-2), severe acute respiratory syndrome (SARS) coronavirus, or Middle East respiratory syndrome (MERS) coronavirus. The coronaviral disease may, for example, be coronavirus disease 19 (COVID-19), severe acute respiratory syndrome (SARS), or Middle East respiratory disease (MERS). Pyroptosis is exploited following infection with SARS-CoV-2, as well as in other coronavirus infections such as Severe acute respiratory syndrome (SARS) and Middle East respiratory syndrome (MERS). Furthermore, COVID-19 patients present a serum biochemistry which indicates high level of cellular pyroptosis.

The disease may be characterised by a biochemistry profile indicative of pyroptosis. The biochemistry profile may, for example, be the biochemistry profile of a sample obtained from the subject. The term “biochemistry profile” may relate to the levels of one or more markers in the sample. Pyroptosis may, for example, be indicated by a biochemistry profile having elevated levels of (i) ASC, (ii) caspase-1, (iii) IL-1B, (iv) IL-18, (v) GSDMD or (vi) HMGB1 in a sample obtained from a subject. For instance, pyroptosis may be indicated by: (i); (ii); (iii); (iv); (v); (vi); (i) and (ii); (i) and (iii); (i) and (iv); (i) and (v); (i) and (vi); (ii) and (iii); (ii) and (iv); (ii) and (v); (ii) and (vi); (iii) and (iv); (iii) and (v); (iii) and (vi); (iv) and (v); (iv) and (vi); (v) and (vi); (i), (ii) and (iii); (i), (ii) and (iv); (i), (ii) and (v); (i), (ii) and (vi); (i), (iii) and (iv); (i), (iii) and (v); (i), (iii) and (vi); (i), (iv) and (v); (i), (iv) and (vi); (i), (v) and (vi); (ii), (iii) and (iv); (ii), (iii) and (v); (ii), (iii) and (vi); (ii), (iv) and (v); (ii), (iv) and (vi); (ii), (v) and (vi); (iii), (iv) and (v); (iii), (iv) and (vi); (iii), (v) and (vi); (iv), (v) and (vi); (i), (ii), (iii) and (iv); (i), (ii), (iii) and (v); (i), (ii), (iii) and (vi); (i), (ii), (iv) and (v); (i), (ii), (iv) and (vi); (i), (ii), (v) and (vi); (i), (iii), (iv) and (v); (i), (iii), (iv) and (vi); (i), (iii), (v) and (vi); (i), (iv), (v) and (vi); (ii), (iii), (iv) and (v); (ii), (iii), (iv) and (vi); (ii), (iii), (v) and (vi); (ii), (iv), (v) and (vi); (iii), (iv), (v) and (vi); (i), (ii), (iii), (iv) and (v); (i), (ii), (iii), (iv) and (vi); (i), (ii), (iii), (v) and (vi); (i), (ii), (iv), (v) and (vi); (i), (iii), (iv), (v) and (vi); (ii), (iii), (iv), (v) and (vi); or (i), (ii), (iii), (iv), (v) and (vi).

An “elevated” level of a marker may, for example, refer to presence of the marker in an increased amount in a sample obtained from an individual of interest compared to the amount of the marker in a sample obtained from a control individual. The control individual may, for example, be a healthy individual. The control individual may, for example, not be afflicted by pyroptosis. Methods for measuring the level of the markers set out above are well-known in the art and may, for example, involve detecting the protein marker or an nucleic acid (e.g. mRNA or DNA) encoding the protein marker. Exemplary techniques may include, western blot coupled to densitometry analyses, or ELISA (to measure protein); RT-PCR (to detect mRNA); or PCR (to detect DNA).

The level of the marker may, for example, be elevated if it is increased by about 1.1 fold or more, for instance at least 2 fold to at least 50 fold, at least 5 fold to at least 40 fold, or at least 10 fold to at least 25 fold relative to the level in a control individual. For example, the level of a marker may be increase by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.1 fold, at least 3.2 fold, at least 3.3 fold, at least 3.4 fold, at least 3.5 fold, at least 3.6 fold, at least 3.7 fold, at least 3.8 fold, at least 3.9 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, at least 5.1 fold, at least 5.2 fold, at least 5.3 fold, at least 5.4 fold, at least 5.5 fold, at least 5.6 fold, at least 5.7 fold, at least 5.8 fold, at least 5.9 fold, at least 6.1 fold, at least 6.2 fold, at least 6.3 fold, at least 6.4 fold, at least 6.5 fold, at least 6.6 fold, at least 6.7 fold, at least 6.8 fold, at least 6.9 fold, at least 7.1 fold, at least 7.2 fold, at least 7.3 fold, at least 7.4 fold, at least 7.5 fold, at least 7.6 fold, at least 7.7 fold, at least 7.8 fold, at least 7.9 fold, at least 8.1 fold, at least 8.2 fold, at least 8.3 fold, at least 8.4 fold, at least 8.5 fold, at least 8.6 fold, at least 8.7 fold, at least 8.8 fold, at least 8.9 fold, at least 9.1 fold, at least 9.2 fold, at least 9.3 fold, at least 9.4 fold, at least 9.5 fold, at least 9.6 fold, at least 9.7 fold, at least 9.8 fold, at least 9.9 fold, at least 10 fold, at least 15 fold, at least 20 fold, at least 25 fold or at least 50 fold relative to the level in a control individual.

A biochemistry profile can be conducted on a sample obtained from the individual of interest. The sample may, for example, comprise a biological fluid such as serum, plasma, whole blood, saliva, sputum, mucus or nasopharyngeal fluid, such as nasopharyngeal mucus. Preferably, the sample comprises whole blood or serum. The biochemistry profile could, for instance, be obtained from a dried blood spot. The biochemistry profile could, for example, be conducted on a nasopharyngeal swab.

The disease may be characterised by increased TSPO expression in a sample obtained from the subject. An increase TSPO expression in the sample may, for example, be relative to TSPO expression in a sample obtained from a control individual. The control individual may, for example, be a healthy individual. The control individual may, for example, not be afflicted by pyroptosis. Methods for measuring expression of a gene product (such as TSPO) are well-known in the art. The measurement of TSPO expression is discussed in more detail below.

TSPO expression may be increased in the sample by about 1.1 fold or more, for instance at least 2 fold to at least 50 fold, at least 5 fold to at least 40 fold, or at least 10 fold to at least 25 fold relative to expression in a control individual. For example, TSPO expression may be increased by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.1 fold, at least 3.2 fold, at least 3.3 fold, at least 3.4 fold, at least 3.5 fold, at least 3.6 fold, at least 3.7 fold, at least 3.8 fold, at least 3.9 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, at least 5.1 fold, at least 5.2 fold, at least 5.3 fold, at least 5.4 fold, at least 5.5 fold, at least 5.6 fold, at least 5.7 fold, at least 5.8 fold, at least 5.9 fold, at least 6.1 fold, at least 6.2 fold, at least 6.3 fold, at least 6.4 fold, at least 6.5 fold, at least 6.6 fold, at least 6.7 fold, at least 6.8 fold, at least 6.9 fold, at least 7.1 fold, at least 7.2 fold, at least 7.3 fold, at least 7.4 fold, at least 7.5 fold, at least 7.6 fold, at least 7.7 fold, at least 7.8 fold, at least 7.9 fold, at least 8.1 fold, at least 8.2 fold, at least 8.3 fold, at least 8.4 fold, at least 8.5 fold, at least 8.6 fold, at least 8.7 fold, at least 8.8 fold, at least 8.9 fold, at least 9.1 fold, at least 9.2 fold, at least 9.3 fold, at least 9.4 fold, at least 9.5 fold, at least 9.6 fold, at least 9.7 fold, at least 9.8 fold, at least 9.9 fold, at least 10 fold, at least 15 fold, at least 20 fold, at least 20 fold or at least and 50 fold in the sample, relative to expression in a control individual.

The sample used to determine TSPO expression may be any biological sample. For example, the sample may comprise a biological fluid such as whole blood, saliva, sputum, mucus or nasopharyngeal fluid, such as nasopharyngeal mucus. The sample may comprise a tissue. Preferably, the sample comprises cells. That is, the sample may comprise a cell sample.

### Individual

The individual may, for example, be a mammal. The mammal may preferably be a human. The mammal may, for example, be a non-human mammal. For instance, the non-human mammal may be a companion or pet animal, such as a dog, cat, rabbit, guinea pig, hamster, ferret, chinchilla, degu, mouse, or rat. The non-human mammal may be an equid, such as a horse or pony. The non-human mammal may be a farm animal, such as an ox, sheep, pig or goat. Alternatively, the individual may be an animal other than a mammal. For instance, the individual may be a bird, such a parrot, chicken, turkey or waterfowl. The individual may be a fish.

The individual may, for example, be an adult. The individual may, for example, be a juvenile.

### Composition

A composition is administered to the individual in order to reduce pyroptosis, and thereby treat or prevent the disease. The composition comprises an agent whose administration reduces TSPO signalling. The agent may, for example, reduce the activity of TSPO, or reduce the amount or accumulation of TSPO.

TSPO (previously known as peripheral benzodiazepine receptor) is a transmembrane protein located on the outer mitochondrial membrane (OMM). Even though TSPO is ubiquitously expressed in mammalian systems, the protein shows a disease-associated pattern of upregulation in chronic conditions (e.g. cancer) and under central nervous system (CNS) inflammation. Previously, the relevance of TSPO signalling to inflammation was unclear, and a role in pyroptosis had not been reported.

As shown in the Examples set out below, the present inventors have demonstrated that TSPO signalling is required for pyroptosis. The inventors have further shown that reduction of TSPO signalling can confer cytoprotective capacity against pyroptosis. In particular, the inventors have shown that TSPO contributes to (i) stabilisation of NLRP3, and (ii) NF-κB retrotranslocation. In doing so, TSPO activates the NLR family pyrin domain containing 3 (NLRP3) inflammasome, an inducer of pyroptosis. The inventors have shown that inhibiting NF-κB nuclear translocation and/or stabilisation of NLRP3 by inhibiting TSPO signalling reduces pyroptosis.

NLRP3 stabilisation contributes to the expression of pro-pyroptotic proteins. NLRP3 is an intracellular sensor that detects a broad range of insults such as microbial motifs, endogenous danger signals and environmental irritants. Such detection results in the formation and activation of the NLRP3 inflammasome, which comprises NLPR3, ASC, and CASP-1. Assembly of the NLRP3 inflammasome leads to caspase 1-dependent release of the pro-inflammatory cytokines IL-1B and IL-18, and recruitment of gasdermin D (GSDMD). IL-1B. IL-18 and GSDMD are mediators of pyroptotic cell death.

NF-κB retrotranslocation to the nucleus also contributes to the expression of pro-pyroptotic proteins. In particular, the inventors have identified that TSPO positively regulates the neuronal apoptosis inhibitor protein (NAIP), MHC class II transactivator type III (C2TA), heterokaryon incompatibility protein E (HET-E), and telomerase-associated protein 1 (TP1 (NACHT), leucine-rich repeat (LRR), and NLR family pyrin domain-containing protein 3 (NLRP3) via NF-κB.

In addition, the inventors have identified that accumulation of dysfunctional mitochondria establishes a molecular platform for prompting both inflammatory signalling and the mitochondrial retrograde response (MRR), and that this platform is lost when TSPO signalling is reduced. In more detail, inhibition of TSPO signalling hampers (i) LPS-mediated mitochondrial depolarization, (ii) oxidative stress and (iii) disruption in mitochondrial functional activity, all of which are crucial triggers of pro-inflammatory cytokine release via pyroptosis.

The inventors are therefore the first to establish that TSPO is a master regulator of pyroptosis, and to recognise that an agent that reduces TSPO signalling can be used to reduce pyroptosis and hence treat associated diseases. In any case, the agent may reduce TSPO signalling by at least 30%. The agent may, for example, reduce TSPO signalling by at least 35%, at least 40%, at least 45%, at least 50%, at least 55%, at least 60%, at least 65%, at least 70%, at least 75%, at least 80%, at least 85%, at least 90%, at least 95%, at least 98%, at least 99%. The agent may, for example, reduce TSPO signalling by 30% to 99%, such as 35% to 95%, 40% to 90%, 45% to 85%, 50% to 80%, 55% to 75%, or 60% to 70%. The agent may, for example, reduce TSPO signalling by 100%. In other words, the agent may completely eliminate the TSPO signalling.

The agent may, for example, reduce the function of TSPO by at least 30%. In other words, the agent may reduce the activity of TSPO by at least 30%. The agent may, for example, reduce the function of TSPO by at least 35%, at least 40%, at least 45%, at least 50%, at least 55%, at least 60%, at least 65%, at least 70%, at least 75%, at least 80%, at least 85%, at least 90%, at least 95%, at least 98%, at least 99%. The agent may, for example, reduce the function of TSPO by 30% to 99%, such as 35% to 95%, 40% to 90%, 45% to 85%, 50% to 80%, 55% to 75%, or 60% to 70%. The agent may, for example reduce the function of TSPO by 100%. In other words, the agent may completely eliminate the function of TSPO. The function of TSPO may be measured by examining effects on molecules downstream of TSPO. For instance, the degree of NLRP3 stabilisation may indicate the function of TSPO. The degree of NF-κB retrotranslocation may indicate the function of TPSO.

The agent may, for example, reduce the expression of TSPO by at least 30%. In other words, the agent may reduce the amount of TSPO mRNA and/or the amount of TSPO protein by at least 30%. The agent may, for example, reduce the expression of TSPO by at least 35%, at least 40%, at least 45%, at least 50%, at least 55%, at least 60%, at least 65%, at least 70%, at least 75%, at least 80%, at least 85%, at least 90%, at least 95%, at least 98%, at least 99%. In other words, the agent may, for example, reduce the amount of TSPO mRNA and/or the amount of TSPO protein by at least 35%, at least 40%, at least 45%, at least 50%, at least 55%, at least 60%, at least 65%, at least 70%, at least 75%, at least 80%, at least 85%, at least 90%, at least 95%, at least 98%, at least 99%. The agent may, for example, reduce the expression of TSPO by 30% to 99%, such as 35% to 95%, 40% to 90%, 45% to 85%, 50% to 80%, 55% to 75%, or 60% to 70%. That is, the agent may, for example, reduce amount of TSPO mRNA and/or the amount of TSPO protein by 30% to 99%, such as 35% to 95%, 40% to 90%, 45% to 85%, 50% to 80%, 55% to 75%, or 60% to 70%. The agent may reduce the expression of TSPO by 100%. In other words, the agent may completely eliminate the expression of TSPO. The agent may, for example, completely eliminate the expression of TSPO mRNA (i.e. reduce the expression of TSPO mRNA by 100%). The agent may, for example, completely eliminate the expression of TSPO protein (i.e. reduce the expression of TSPO protein by 100%). The agent may, for example, completely eliminate the expression of TSPO mRNA and TSPO protein (i.e. reduce the expression of TSPO mRNA and TSPO protein by 100%).

Reduction or elimination of TSPO signalling may in turn reduce activation of the NLRP3 inflammasome. Activation of the NLRP3 inflammasome may, for example, be reduced by reducing and/or counteracting TSPO-mediated NRLP3 stabilisation. Activation of the NLRP3 inflammasome may, for example, be reduced by reducing and/or counteracting TSPO-mediated NF-κB nuclear translocation. As a result of reduced activation of the NLRP3 inflammasome, the expression of pyroptotic proteins (such as ASC, caspase-1, IL-1B, IL-18, GSDMD and/or HMGB1) may be reduced.

The agent may, therefore reduce activation of the NLRP3 inflammasome. The agent may reduce TSPO-mediated NRLP3 stabilisation. The agent may reduce TSPO-mediated NF-κB nuclear translocation. The agent may reduce the expression of pyroptotic proteins, such as ASC, caspase-1, IL-1B, IL-18, GSDMD and/or HMGB1. The agent may, for example, reduce the activation of the NLRP3 inflammasome, TSPO-mediated NRLP3 stabilisation, TSPO-mediated NF-κB nuclear translocation, and/or the expression of pyroptotic proteins by at least 30%. The agent may, for example, reduce the activation of the NLRP3 inflammasome, TSPO-mediated NRLP3 stabilisation, TSPO-mediated NF-κB nuclear translocation, and/or the expression of pyroptotic proteins by at least 35%, at least 40%, at least 45%, at least 50%, at least 55%, at least 60%, at least 65%, at least 70%, at least 75%, at least 80%, at least 85%, at least 90%, at least 95%, at least 98%, at least 99%. The agent may, for example, reduce the activation of the NLRP3 inflammasome, TSPO-mediated NRLP3 stabilisation, TSPO-mediated NF-κB nuclear translocation, and/or the expression of pyroptotic proteins by 30% to 99%, such as 35% to 95%, 40% to 90%, 45% to 85%, 50% to 80%, 55% to 75%, or 60% to 70%. The agent may reduce the activation of the NLRP3 inflammasome. TSPO-mediated NRLP3 stabilisation, TSPO-mediated NF-κB nuclear translocation, and/or the expression of pyroptotic proteins by 100%. In other words, the agent may completely eliminate the activation of the NLRP3 inflammasome, TSPO-mediated NRLP3 stabilisation, TSPO-mediated NF-κB nuclear translocation, and/or the expression of pyroptotic proteins.

Agents that reduce TSPO signalling are known in the art. The agent may, for example, be a TSPO ligand. The agent may, for example, be a nucleic acid silencing molecule that reduces the expression of TSPO. The agent may, for example, be an antibody, a polypeptide, an polynucleotide, a polyribonucleotide, a lipid, a nucleic acid, a ribonucleic acid, an amino acid, a carbohydrate, a fatty acid, a vitamin, an organic compound, an inorganic compound. Any of these agents may be capable of reducing TSPO signalling.

The term “TSPO ligand” is a term of art that refers to TSPO-binding molecules, such as TSPO-binding drugs. Binding of a TSPO ligand to TSPO may reduce downstream signalling. For example, binding of a TSPO ligand to TSPO may reduce NLRP3 stablisation and/or NF-κB retrotranslocation. In other words, a TSPO ligand may reduce the function of TSPO. Numerous TSPO ligands are known in the art. Both synthetic and non-synthetic TSPO ligand are known. A non-synthetic TSPO ligand may, for example, be an endogenously produced TSPO ligand. Such a TSPO ligand may be used therapeutically to reduce the expression of TSPO. The agent may, therefore be a non-synthetic TSPO ligand. Preferably, though, the agent is a synthetic TSPO ligand. For example, the TSPO ligand may be etifoxine, PK11195, XBD173, FGIN, or SSR-180,575. The composition may comprise one or more of (i) etifoxine, (ii) PK11195, (iii) XBD173, (iv) FGIN, and (v) SSR-180,575 in any combination, such as: (i); (ii); (iii); (iv); (v); (i) and (ii); (i) and (iii); (i) and (iv); (i) and (v); (ii) and (iii); (ii) and (iv); (ii) and (v); (iii) and (iv); (iii) and (v); (iv) and (v); (i), (ii) and (iii); (i), (ii) and (iv); (i), (ii) and (v); (i), (iii) and (iv); (i), (iii) and (v); (i), (iv) and (v); (ii), (iii) and (iv); (ii), (iii) and (v); (ii), (iv) and (v); (iii), (iv) and (v); (i), (ii), (iii) and (iv); (i), (ii), (iii) and (v); (i), (ii), (iv) and (v); (i), (iii), (iv) and (v); (ii), (iii), (iv) and (v); or (i), (ii), (iii), (iv) and (v).

A nucleic acid silencing molecule that reduces the expression of TSPO may reduce or eliminate expression of TSPO. The nucleic acid silencing molecule may, for example, reduce the amount of the mRNA product of the TSPO gene. The nucleic acid silencing molecule may, for example, eliminate the mRNA product of the TSPO gene. The nucleic acid silencing molecule may, for example, reduce the amount of the protein product of the TSPO gene. The nucleic acid silencing molecule may, for example, eliminate the protein product of the TSPO gene.

The nucleic acid silencing molecule may be defined as a silencing molecule that comprises or consists of one or more nucleic acids. The nucleic acid silencing molecule may itself be a nucleic acid. The nucleic acid silencing molecule of the disclosure may comprise RNA. The nucleic acid silencing molecule of the disclosure may comprise DNA. The nucleic acid silencing molecule of the disclosure may comprise DNA and RNA. The nucleic acid silencing molecule of the disclosure may consist of RNA. The nucleic acid silencing molecule of the disclosure may consist of DNA. The nucleic acid silencing molecule of the disclosure may consist of DNA and RNA.

The nucleic acid silencing molecule may reduce or eliminate (i.e. knock down) expression of TSPO by any mechanism known in the art. The nucleic acid silencing molecule may, for example, bind to a mRNA molecule encoded by the TSPO gene to block its translation into protein. The nucleic acid silencing molecule may, for example, bind to a mRNA molecule encoded by the TSPO gene to induce degradation (such as enzymatic degradation) of the mRNA. The nucleic acid silencing molecule may, for example, bind to DNA encoding the TSPO gene to induce methylation of the DNA and/or its associated histones. The nucleic acid silencing molecule may, for example, bind to DNA encoding TSPO to facilitate removal of all or part of the TSPO gene by gene editing.

For example, the nucleic acid silencing molecule may comprise or consist of an antisense oligonucleotide (AON). The nucleic acid silencing molecule may comprise or consist of a small interfering RNA (siRNA). The nucleic acid silencing molecule may comprise or consist of a short hairpin RNA (shRNA). The nucleic acid silencing molecule may comprise or consist of a microRNA (miRNA). The nucleic acid silencing molecule may comprise or consist of a CRISPR guide RNA. In this case, a CRISPR nuclease (such as Cas9, Cpf1, Cas12b, or CasX) may also be administered to the subject. The CRISPR nuclease may be comprised in the composition comprising the nucleic acid silencing molecule, or in a separate composition.

The nucleic acid silencing molecule may be about 10 to about 15000 nucleotides in length, such as about 100 to about 14000, about 200 to about 13000, about 300 to about 12000, about 400 to about 11000, about 400 to about 10000, about 500 to about 9000, about 600 to about 8000, about 700 to about 7000, about 800 to about 6000, about 900 to about 5000, about 1000 to about 4000, or about 2000 to 3000 in length. Preferably, the nucleic acid silencing molecule is less than 100 (such as less than 95, less than 90, less than 85, less than 80, less than 75, less than 70, less than 65, less than 60, less than 55, less than 50, less than 45, less than 40, less than 35, less than 30, less than 25, less than 20, less than 15, or less than 10) nucleotides in length. The nucleic acid silencing molecule may, for example be about 10 to about 50 nucleotides in length. For example, the nucleic acid silencing molecule may be about 10 to about 40, about 10 to about 30, about 10 to about 20, about 20 to about 50, about 20 to about 40, about 20 to about 30, about 30 to about 50, or about 30 to about 40 nucleotides in length. Preferably, the nucleic acid molecule is about 10 to about 30 (such as about 10 to about 20, or about 20 to about 30) nucleotides in length. The nucleic acid molecule may, for example, be about 10, about 11, about 12, about 13, about 14, about 14, about 16, about 17, about 18, about 19, about 20, about 21, about 22, about 23, about 24, about 25, about 26, about 27, about 28, about 29 or about 30 nucleotides in length. The nucleic acid molecule may preferably be about 16 or about 20 nucleic acids in length. Typical lengths of antisense oligonucleotides (AONs), small interfering RNAs (siRNAs), short hairpin RNAs (shRNAs), microRNAs (miRNAs), and CRISPR guide RNAs are well-known in the art.

The nucleic acid silencing molecule may, for example, comprise one or more 2′-O-methoxyethylribose (MOE) modified nucleotides or consist of 2′-O-methoxyethylribose (MOE) modified nucleotides. The nucleic acid silencing molecule may, for example, comprise one or more 2′-O-methyl (2OMe) modified nucleotides or consist of 2′-O-methyl (2OMe) modified nucleotides. The nucleic acid silencing molecule may, for example, comprise one or more locked nucleic acid (LNA) modified nucleotides or consist of locked nucleic acid (LNA) modified nucleotides. The nucleic acid silencing molecule may, for example, comprise one or more nucleotide phosphorothioates or consist of nucleotide phosphorothioates. MOE modified nucleotides, 2OMe modified nucleotides, LNA modified nucleotides and nucleotide phosphorothioates are described in the art.

The nucleic acid silencing molecule may comprise any combination of (i) one or more 2OMe modified nucleotides, (ii) one or more LNA modified nucleotides, (iii) one or more 2′-O-methoxyethylribose (MOE) modified nucleotides and (iv) one or more nucleotide phosphorothioates. For example, the nucleic acid silencing molecule may comprise (i); (ii); (iii); (iv); (i) and (ii); (i) and (iii); (i) and (iv); (ii) and (iii); (ii) and (iv); (iii) and (iv); (i), (ii) and (iii); (i), (ii) and (iv); (i), (iii) and (iv); (ii), (iii) and (iv); or (i), (ii), (iii) and (iv).

The nucleic acid silencing molecule may be capable of binding to the TSPO gene or to the RNA encoded by the TSPO gene. The nucleic acid silencing molecule may be capable of binding to part of the TSPO gene or to part of the RNA encoded by the TSPO gene. Binding may, for example, be effected by hybridisation.

The nucleic acid silencing molecule may be directed to the nucleic acid sequence of the TSPO gene. A nucleic acid silencing molecule that is “directed to” a particular nucleic acid sequence is capable of binding to (e.g. hybridising to) that nucleic acid sequence. For example, the nucleic acid silencing molecule may be directed to the DNA of SEQ ID NO: 1. The nucleic acid silencing molecule may be directed to RNA encoded by the TSPO gene. For example, the nucleic acid silencing molecule may be directed to mRNA encoded by the TSPO gene. The nucleic acid silencing molecule may be directed to RNA encoded by the DNA of SEQ ID NO: 1.

The nucleic acid silencing molecule may be directed to a nucleic acid sequence encoding TSPO protein. For example, the nucleic acid silencing molecule may be directed to a nucleic acid sequence encoding the TSPO protein of SEQ ID NO: 2. The nucleic acid silencing molecule may be directed to nucleic acid sequence encoding a protein having at least 90% sequence identity to SEQ ID NO: 2. For instance, the nucleic acid silencing molecule may be directed to nucleic acid sequence encoding a protein having at least 91%, at least 92%, at least 93%, at least 94%, at least 95%, at least 96%, at least 97%, at least 98%, or at least 99% sequence identity to SEQ ID NO: 2.

The nucleic acid silencing molecule may comprise (a) a nucleotide sequence that has at least 75% sequence identity to a nucleotide sequence comprised in a primary transcript of TSPO or (b) a nucleotide sequence that is complementary to the nucleotide sequence of (a). The nucleic acid silencing molecule may, for example, comprise or consist of an antisense oligonucleotide (AON). In one aspect, the nucleic acid silencing molecule may comprise a nucleotide sequence that has at least 80%, at least 85%, at least 90%, at least 95%, at least 96%, at least 97%, at least 98%, at least 99%, or 100% sequence identity to a nucleotide sequence comprised in a primary transcript of TSPO. In one aspect, the nucleic acid silencing molecule may comprise a nucleotide sequence that has 80% to 100%, 85% to 99%, 90% to 98%, or 95% to 97% sequence identity to a nucleotide sequence comprised in a primary transcript of TSPO. In one aspect, the nucleic acid silencing molecule may comprise a nucleotide sequence that is complementary to a nucleotide sequence that has at least 80%, at least 85%, at least 90%, at least 95%, at least 96%, at least 97%, at least 98%, at least 99%, or 100% sequence identity to a nucleotide sequence comprised in a primary transcript of TSPO. In one aspect, the nucleic acid silencing molecule may comprise a nucleotide sequence that is complementary to a nucleotide sequence that has 80% to 100%, 85% to 99%, 90% to 98%, or 95% to 97% sequence identity to a nucleotide sequence comprised in a primary transcript of TSPO. A primary transcript is the single-stranded ribonucleic acid RNA product synthesised by transcription of DNA, that is processed to yield various mature RNA products such as mRNAs, tRNAs, and rRNAs. The primary transcript may, for example, be a precursor mRNA (pre-mRNA) that is processed to form mRNA. The nucleotide sequence comprised in the primary transcript may comprise one or more of (i) a nucleotide sequence comprised in an exon, (ii) a nucleotide sequence comprised in an intron, (iii) a nucleotide sequence comprised in a 3′ untranslated region, and (iv) a nucleotide sequence comprised in a 5′ untranslated region. The nucleotide sequence comprised in the primary transcript may, for example, comprise: (i); (ii); (iii); (iv); (i) and (ii); (i) and (iii); (i) and (iv); (ii) and (iii); (ii) and (iv); (iii) and (iv); (i), (ii) and (iii); (i), (ii) and (iv); (i), (iii) and (iv); (ii), (iii) and (iv); or (i), (ii), (iii) and (iv).

The nucleic acid silencing molecule may comprise (a) a nucleotide sequence that has at least 75% sequence identity to a nucleotide sequence comprised in a mRNA transcribed from TSPO or (b) a nucleotide sequence that is complementary to the nucleotide sequence of (a). The nucleic acid silencing molecule may, for example, comprise or consist of an antisense oligonucleotide (AON). The nucleic acid silencing molecule may, for example, comprise or consist of a small interfering RNA (siRNA). In one aspect, the nucleic acid silencing molecule may comprise a nucleotide sequence that has at least 80%, at least 85%, at least 90%, at least 95%, at least 96%, at least 97%, at least 98%, at least 99%, or 100% sequence identity to a nucleotide sequence comprised in a mRNA transcribed from TSPO. In one aspect, the nucleic acid silencing molecule may comprise a nucleotide sequence that has 80% to 100%, 85% to 99%, 90% to 98%, or 95% to 97% sequence identity to a nucleotide sequence comprised in a mRNA transcribed from TSPO. In one aspect, the nucleic acid silencing molecule may comprise a nucleotide sequence that is complementary to a nucleotide sequence that has at least 80%, at least 85%, at least 90%, at least 95%, at least 96%, at least 97%, at least 98%, at least 99%, or 100% sequence identity to a nucleotide sequence comprised in a mRNA transcribed from TSPO. In one aspect, the nucleic acid silencing molecule may comprise a nucleotide sequence that is complementary to a nucleotide sequence that has 80% to 100%, 85% to 99%, 90% to 98%, or 95% to 97% sequence identity to a nucleotide sequence comprised in a mRNA transcribed from TSPO.

The siRNA may comprise a sequence defined by SEQ ID NO:7, SEQ ID NO:8 or SEQ ID NO:9. The siRNA may comprise a sequence that has one or more (such as two or more, three or more, four or more or five or more) amino acid mutations relative to SEQ ID NO:7. The siRNA may comprise a sequence that has such as one or more (such as two or more, three or more, four or more or five or more) amino acid mutations relative to SEQ ID NO:8. The siRNA may comprise a sequence that has one or more (such as two or more, three or more, four or more or five or more) amino acid mutations relative to SEQ ID NO:9. Each amino acid mutation may be independently selected from a substitution, an insertion, and a deletion. An amino acid substitution may, for example, be a conservative amino acid substitution. Conservative amino acid substitutions are defined in detail below. If the agent is a nucleic acid silencing molecule, the composition may comprise a delivery vehicle that optimises delivery of the nucleic acid silencing molecule in vivo. Suitable delivery vehicles are known in the art and include, for example, cell-targeting moieties, cell-penetrating moieties, lipids, lipoproteins, liposomes, lipoplexes, peptides, GalNAc, antibodies, aptamers, nanoparticles, exosomes, spherical nucleic acids, and DNA cages.

In any case, the composition may comprise a physiologically acceptable carrier or diluent in addition to the agent. Typically, such compositions are prepared as liquid suspensions. The agent may, for example, be mixed with an excipient which is pharmaceutically acceptable and compatible with the active ingredient. Suitable excipients are, for example, water, saline, dextrose, glycerol, of the like and combinations thereof. In addition, if desired, the pharmaceutical compositions may contain minor amounts of auxiliary substances such as wetting or emulsifying agents, and/or pH buffering agents.

### Administration

The composition may be administered by any route. Suitable routes include, but are not limited to, the intravenous, intrathecal, intracerebral ventricular, intramuscular, intraperitoneal, subcutaneous, intradermal, transdermal and oral/buccal routes.

The composition is administered in a manner compatible with the dosage formulation of the agent and in such amount will be therapeutically effective. The quantity to be administered depends on the subject to be treated, the disease to be treated, and the capacity of the individual's immune system. Precise amounts of agents required to be administered may depend on the judgement of the practitioner and may be peculiar to each subject.

For instance, the composition may comprise one or more nucleic acid signalling molecules that reduce the expression of TSPO. For example, the composition may comprise two or more, five or more, ten or more, 20 or more, 50 or more, 100 or more, 200 or more, 500 or more, 1000 or more, 2000 or more, 5000 or more, 10000 or more, 20000 or more, 50000 or more, 100000 or more, 200000 or more, 500000 or more, 1000000 or more, 2000000 or more, 5000000 or more, 1×10⁷ or more, 2×10⁷ or more, 5×10⁷ or more, 1×10⁸ or more, 2×10⁸ or more, 5×10⁸ or more, 1×10⁹ or more, 2×10⁹ or more, or 5×10⁹ or more nucleic acid silencing molecules that reduce the expression of TSPO per dose.

The composition may comprise one or more TSPO ligands. For example, the composition may comprise two or more, five or more, ten or more, 20 or more, 50 or more, 100 or more, 200 or more, 500 or more, 1000 or more, 2000 or more, 5000 or more, 10000 or more, 20000 or more, 50000 or more, 100000 or more, 200000 or more, 500000 or more, 1000000 or more, 2000000 or more, 5000000 or more, 1×10⁷ or more, 2×10⁷ or more, 5×10⁷ or more, 1×10⁸ or more, 2×10⁸ or more, 5×10⁸ or more, 1×10⁹ or more, 2×10⁹ or more, or 5×10⁹ or more TSPO ligands per dose.

### Combination Therapy

The composition may be administered as part of a combination therapy. That is, the method of treatment or medical use may comprise administering to the subject a further therapeutic composition. Administration of a further composition may, for example, be desirable when the agent reduces rather than eliminates TSPO expression. In some cases, though, reduction (rather than elimination) of TSPO expression may be sufficient to effect treatment of the disease.

The composition may be administered as part of a combination therapy in conjunction with any available therapeutic composition for a particular disorder. For example, the composition may be administered together with any treatments for COVID-19 such as antibody-based therapies.

In one aspect of combination therapy, the composition that an agent that reduces the TSPO signalling is be administered in such an amount that will be therapeutically effective in combination with administration of the further therapeutic composition. The further therapeutic composition may be administered in such an amount that will be therapeutically effective in combination with the composition that comprises an agent that reduces TSPO signalling.

The composition that comprises an agent that reduces TSPO signalling and the further therapeutic composition may be administered together, for instance at the same time. The composition that comprises an agent that reduces TSPO signalling and the further therapeutic composition may be administered separately, for instance at a different time. For example, the composition that comprises an agent that reduces TSPO signalling may be administered before the further therapeutic composition. The composition that comprises an agent that reduces TSPO signalling may be administered after the further therapeutic composition. Administration of the composition that comprises an agent that reduces TSPO signalling may be alternated with administration of the further therapeutic composition.

### Use of TSPO Expression as a Biomarker for Pyroptosis

The disclosure provides use of TSPO expression as a biomarker for pyroptosis. As discussed above and demonstrated in the Examples, TSPO is overexpressed in diseases associated with pyroptosis, such as viral (e.g. SARS-CoV-2) infections. Furthermore, reduction of TSPO expression and/or function protects against pyroptosis. It is clear, therefore, that TSPO expression is correlated with pyroptosis. That is, TSPO expression is associated with pyroptosis. TSPO expression can be used as a biomarker for pyroptosis.

Use of TSPO expression as a biomarker for pyroptosis may, for example, involve measuring the amount of TSPO expression. TSPO expression may, for example, be measured in a sample. The sample may comprise any biological sample. For example, the sample may comprise a biological fluid such as whole blood, saliva, sputum, mucus or nasopharyngeal fluid. The sample may comprise a tissue. Preferably, the sample comprises cells. That is, the sample may comprise a cell sample. The sample, may, for instance, comprise a sample obtained from an individual. The individual may be an individual of interest. For example, the individual may be an individual suspected of having pyroptosis.

Any method may be used to measure TSPO expression. Methods for measuring expression of a protein, such as TSPO, are well-known in the art. TSPO expression may, for example, be determined by measuring the amount of TSPO protein. The amount of TSPO protein may, for example, be measured using Western blotting, an enzyme-linked immunosorbent assay ELISA), protein immunoprecipitation, immunoelectrophoresis, protein immunostaining, high-performance liquid chromatography (HPLC) or liquid chromatography-mass spectrometry (LC-MS). TSPO expression may, for example, be determined by measuring the amount of TSPO mRNA. The amount of TSPO mRNA may, for example, be measured using Northern blotting, a nuclease protection assay (NPA), in situ hybridization, or reverse transcription-polymerase chain reaction (RT-PCR).

A low level of TSPO expression may indicate the absence of pyroptosis, or a low level of pyroptosis. A high level of TSPO expression may indicate the presence of pyroptosis, or a high level of pyroptosis. Measured TSPO expression may, for example, compared to a reference value. TSPO expression less than or the same as the reference value may indicate the absence of pyroptosis, or a low level of pyroptosis. In the context of the disclosure, a low level of pyroptosis may mean that the level of pyroptosis is not clinically significant. TSPO expression more than the reference value may indicate the presence of pyroptosis, or a high level of pyroptosis. In the context of the disclosure, a high level of pyroptosis may mean that the level of pyroptosis is clinically significant. The reference value may, for example, reflect the level of TSPO expression in one or more healthy individuals. For instance, the reference value may reflect the average level of TSPO expression in healthy individuals. A healthy individual may be defined as an individual not experiencing clinically significant pyroptosis.

TSPO expression in a sample obtained from and individual may be compared to a control. The control may, for example, be the level of TSPO expression in a sample obtained from a healthy individual (such as an individual not afflicted by clinically significant pyroptosis). Increased TSPO expression relative to the control may, for instance, indicate increased pyroptosis in the individual. Increased pyroptosis may, for example, be clinically significant. For instance, the individual may for example, have a disease characterised by pyroptosis. Decreased TSPO expression relative to the control, or TSPO expression equivalent to the control, may indicate that pyroptosis is not increased in the individual. For instance, the individual may have a level of pyroptosis that is not clinically significant, or pyroptosis may be absent in the individual.

### Method of Assessing the Degree of Pyroptosis

The disclosure provides a method of assessing the degree of pyroptosis in an individual. The method may be practiced in vitro. The method comprises measuring the amount of TSPO expressed in a sample obtained from the individual. The amount of TSPO expressed in the sample correlates with the degree of pyroptosis in the individual. In other words, the amount of TSPO expressed in the sample is associated with, and/or indicates, the degree of pyroptosis in the individual. In essence, the method relies on use of TSPO expression as a biomarker for pyroptosis. Such use is described in detail above.

Any of the aspects described in connection with use of TSPO expression as a biomarker for pyroptosis may also apply to the method of assessing the degree of pyroptosis. For instance, TSPO can be measured as described above. The sample may be any of the sample types described above.

In the context of the present disclosure, the “degree of pyroptosis” may refer to the amount or level of pyroptosis present in the individual. The level of TSPO measured in the sample may indicate the level of pyroptosis in the individual. That is, the amount of TSPO measured in the sample may indicate the amount of pyroptosis in the individual. In other words, the degree of pyroptosis may be indicated by the measured amount or level of TSPO. A low level, amount or degree of pyroptosis may, for example, mean that little or no pyroptosis is occurring in the individual. For instance, the level or pyroptosis may not be clinically significant. A high level, amount or degree of pyroptosis may mean that pyroptosis is occurring in the individual, for instance in an amount greater than normal. For instance, the level or pyroptosis may be clinically significant. In this context “normal” may mean the amount of pyroptosis in one or more healthy individuals, such as the average amount of pyroptosis in healthy individuals.

### In Vitro Model of Pyroptosis

The disclosure provides an in vitro model of pyroptosis. The in vitro model comprises cells modified to comprise a polynucleotide encoding one or more virus-related proteins that activate the NLRP3 inflammasome. The actual overexpression of TSPO is contemplated to be a model to trigger pyroptosis per se.

As discussed above, activation of the NLRP3 inflammasome drives pyroptosis.

### Cells

The cells used in the in vitro model may comprise any cells capable of modification to comprises a polynucleotide encoding one or more virus-related proteins. In essence, the cells may comprise any cells that may express a protein.

The cells may, for example, comprise mammalian, bacterial, plant and/or yeast cells. For example, the mammalian cells may be human cells. The cells may comprise a cell line, for instance a human cell line. The cells may, for example, comprise monocytes. The monocytes may, for instance, comprise THP-1 cells. The cells may, for example, comprise lung cells. The lung cells may, for instance, comprise A549 cells. The cells may, for example, comprise epithelial cells. The epithelial cells may, for instance, comprise Vero cells.

The cells are modified to comprise a polynucleotide encoding one or more virus-related proteins that activate the NLRP3 inflammasome. In other words, the cells may be modified cells. Polynucleotides and proteins are described in detail below. Modification may, for example, be effected by transfection or transformation with the polynucleotide. For example, the cell may be transfected using PEI (polyethylenimine)-based transfection reagents or by electroporation.

In a preferred aspect, the cells express the one or more virus-related proteins encoded by the polynucleotide. For example, the cells may transiently express the one or more virus related proteins. The cells may stably express the one or more virus related. For example, the cells may comprise a stable cell line.

### Polynucleotide

The cells are modified to comprise a polynucleotide encoding one or more virus related-proteins that activate the NLRP3 inflammasome. The cells may, for example, be modified to comprise two or more, such as three or more, four or more, or five our more, polynucleotide each encoding one or more virus-related proteins that activate the NLRP3 inflammasome.

The polynucleotide may, for example, comprise DNA, RNA, PNA, GNA, TNA LNA, HNA and/or XNA. Preferably, the polynucleotide comprises DNA and/or RNA.

The polynucleotide may, for example, be comprised in a vector, such as a viral vector. The polynucleotide may, for example, integrate into the genome of the cell.

The polynucleotide may, for example, comprise one or more sequence other than that encoding the one or more virus-related proteins. For instance, the polynucleotide may comprise a promoter sequence. The sequence encoding the one or more virus-related proteins may be operably linked to the promoter sequence.

### Protein

The polynucleotide encodes one or more virus-related proteins that activate the NLRP3 inflammasome. The polynucleotide may, for example, encode two or more (such as three or more, four or more, or five or more) such proteins.

The one or more proteins may be derived from any virus. That is, the one or more proteins may be related to any virus. In other words, the one or more proteins may, in nature, be expressed by any virus. When the polynucleotide encodes two or more virus-related proteins, each protein may be derived from the same virus or a different virus. When the polynucleotide encodes three or more virus-related proteins, each protein may be derived from the same virus. Each of the three or more proteins may be derived from a different virus. Some of the three or more proteins may be derived from the same virus, and some of the three or more proteins may be derived from a different virus.

In one aspect, the virus may be a coronavirus. Numerous coronaviruses are known in the art, and the virus may be any thereof. Coronaviruses are known inducers of pyroptosis. Preferably, the coronavirus is a human coronavirus, such as SARS-CoV-2. SARS-CoV, MERS-COV, or a common cold coronavirus. More preferably, the virus is SARS-CoV-2.

The protein may be any protein expressed by the virus, providing that it activates the NLRP3 inflammasome. Methods for measuring activation of the NLRP3 inflammasome are known in the art. Thus, the skilled person can readily identify virus-related proteins possessing the desired function.

When the virus is SARS-CoV-2, the one or more virus-related proteins may comprise all or part of ORF 8.2b. The one or more virus-related proteins may comprise all or part ORF 3a. The one or more virus-related proteins may comprise all or part of ORF 8.2b and all or part of ORF 3a. The amino acid sequence of ORF 8.2b may be represented by SEQ ID NO: 3. The polynucleotide sequence encoding ORF 8.2b may be represented by SEQ ID NO: 4. The amino acid sequence of ORF 3a may be represented by SEQ ID NO: 5. The polynucleotide sequence encoding ORF 3a may be represented by SEQ ID NO: 6. Part of ORF 8.2b or ORF 3a may refer to a peptide derived from ORF 8.2b or ORF 3a respectively. The peptide may, for example, be from 5 to 100 amino acids in length, such as from 5 to 10, 10 to 25, 10 to 90, 20 to 80, 30 to 70, or 40 to 60 amino acids in length. For instance, the peptide may be at least 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or 95 amino acids in length.

Mechanistically, expression of the Open Reading Frame (ORF) (e.g. 8.2b, 3a) by the SARS-CoV-2 virus impairs functions of organelles determinant of cellular bio-energy and signalling such as the endoplasmic reticulum and mitochondria. This leads to the activation of the NLRP3 inflammasome which triggers the inflammatory cellular response causing premature and detrimental demise.

Other virus-related proteins that may trigger the same response include the SARS-CoV-2 nucleocapsid (N) protein and the SARS-CoV-2 Envelope (E) protein. The one or more virus-related proteins thus may comprise all or part of the SARS-CoV-2 nucleocapsid (N) protein. The one or more virus-related proteins may thus comprise all or part of the SARS-CoV-2 nucleocapsid (E) protein. The amino acid sequence of N protein may be represented by SEQ ID NO: 10. The polynucleotide sequence encoding N protein may be represented by SEQ ID NO: 11. The amino acid sequence of E protein may be represented by SEQ ID NO: 12. The polynucleotide sequence encoding E protein may be represented by SEQ ID NO: 13. Part of N protein or E protein may refer to a peptide derived from N protein or E protein respectively. The peptide may, for example, be from 5 to 100 amino acids in length, such as from 5 to 10, 10 to 25, 10 to 90, 20 to 80, 30 to 70, or 40 to 60 amino acids in length. For instance, the peptide may be at least 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or 95 amino acids in length.

The one or more virus-related proteins may comprise a protein having at least 70% identity to ORF 8.2b (SEQ ID NO: 3). For instance, the one or more virus-related proteins may comprise a protein having at least 75%, at least 80%, at least 85%, at least 90%, at least 95%, at least 96%, at least 97%, at least 98% or at least 99% identity to ORF 8.2b (SEQ ID NO: 3). The one or more virus-related proteins may comprise a protein encoded by a polynucleotide sequence having at least 70% identity to SEQ ID NO: 4. For instance, the one or more virus-related proteins may comprise a protein encoded by a polynucleotide sequence having at least 75%, at least 80%, at least 85%, at least 90%, at least 95%, at least 96%, at least 97%, at least 98% or at least 99% identity to SEQ ID NO: 4. The one or more virus-related proteins may comprise a protein having at least 70% identity to ORF 3a (SEQ ID NO: 5). For instance, the one or more virus-related proteins may comprise a protein having at least 75%, at least 80%, at least 85%, at least 90%, at least 95%, at least 96%, at least 97%, at least 98% or at least 99% identity to ORF 3a (SEQ ID NO: 5). The one or more virus-related proteins may comprise a protein encoded by a polynucleotide sequence having at least 70% identity to SEQ ID NO: 6. For instance, the one or more virus-related proteins may comprise a protein encoded by a polynucleotide sequence having at least 75%, at least 80%, at least 85%, at least 90%, at least 95%, at least 96%, at least 97%, at least 98% or at least 99% identity to SEQ ID NO: 6. The one or more virus-related proteins may comprise a protein having at least 70% identity to SARS-CoV-2 N protein (SEQ ID NO: 10). For instance, the one or more virus-related proteins may comprise a protein having at least 75%, at least 80%, at least 85%, at least 90%, at least 95%, at least 96%, at least 97%, at least 98% or at least 99% identity to SARS-CoV-2 N protein (SEQ ID NO: 10). The one or more virus-related proteins may comprise a protein encoded by a polynucleotide sequence having at least 70% identity to SEQ ID NO: 11. For instance, the one or more virus-related proteins may comprise a protein encoded by a polynucleotide sequence having at least 75%, at least 80%, at least 85%, at least 90%, at least 95%, at least 96%, at least 97%, at least 98% or at least 99% identity to SEQ ID NO: 11. The one or more virus-related proteins may comprise a protein having at least 70% identity to SARS-CoV-2 E protein (SEQ ID NO: 12). For instance, the one or more virus-related proteins may comprise a protein having at least 75%, at least 80%, at least 85%, at least 90%, at least 95%, at least 96%, at least 97%, at least 98% or at least 99% identity to SARS-CoV-2 N protein (SEQ ID NO: 12). The one or more virus-related proteins may comprise a protein encoded by a polynucleotide sequence having at least 70% identity to SEQ ID NO: 13. For instance, the one or more virus-related proteins may comprise a protein encoded by a polynucleotide sequence having at least 75%, at least 80%, at least 85%, at least 90%, at least 95%, at least 96%, at least 97%, at least 98% or at least 99% identity to SEQ ID NO: 13.

The one or more virus-related proteins may differ from ORF 8.2b, ORF 3a, N protein or E protein by one or more amino acid mutations. For instance, the virus-related protein may differ from ORF 8.2b, ORF 3a, N protein or E protein by two or more, three or more, four or more, five or more, six or more, seven or more, eight or more, nine or more, or ten or more amino acid mutations. The one or more mutations may include one or more amino acid additions, on or more amino acid deletions, and/or one or more amino acid substitutions made relative to ORF 8.2b, ORF 3a. N protein or E protein. The substitutions may be conservative or non-conservative amino acid substitutions. Where there are multiple substitutions in a single polypeptide, the modifications in the polypeptide sequence may be a combination of conservative and non-conservative amino acid substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in the Table below.

TABLE 1
Chemical properties of amino acids

| | | | |
|---|---|---|---|
| Ala (A) | aliphatic, hydrophobic, neutral | Met (M) | hydrophobic, neutral |
| Cys (C) | polar, hydrophobic, neutral | Asn (N) | polar, hydrophilic, neutral |
| Asp (D) | polar, hydrophilic, charged (−) | Pro (P) | hydrophobic, neutral |
| Glu (E) | polar, hydrophilic, charged (−) | Gln (Q) | polar, hydrophilic, neutral |
| Phe (F) | aromatic, hydrophobic, neutral | Arg (R) | polar, hydrophilic, charged |
| | | | (+) |
| Gly (G) | aliphatic, neutral | Ser (S) | polar, hydrophilic, neutral |
| His (H) | aromatic, polar, hydrophilic, | Thr (T) | polar, hydrophilic, neutral |
| | charged (+) | | |
| Ile (I) | aliphatic, hydrophobic, neutral | Val (V) | aliphatic, hydrophobic, |
| | | | neutral |
| Lys (K) | polar, hydrophilic, charged(+) | Trp (W) | aromatic, hydrophobic, |
| | | | neutral |
| Leu (L) | aliphatic, hydrophobic, neutral | Tyr (Y) | aromatic, polar, hydrophobic |

Where amino acids have similar polarity, this can be determined by reference to the hydropathy scale for amino acid side chains in the Table below.

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

### Vector

The disclosure provides a vector comprising a polynucleotide that encodes one or more virus-related proteins that activate the NLRP3 inflammasome. Such polynucleotide and virus-related proteins are described above in connection with the in vitro model of the disclosure. Any of the aspects disclosed in connection with the in vitro model of the disclosure may also apply to the vector.

In one aspect, the vector comprises a polynucleotide encoding ORF 8.2b and/or ORF 3a from SARS-CoV2. For instance, the vector may comprise a polynucleotide encoding ORF 8.2b. The vector may comprise a polynucleotide encoding ORF 3a. The vector may comprise a polynucleotide encoding ORF 8.2b and ORF 3a.

The vector disclosed herein may be any vector known in the art. The vector may, for example, be a viral vector. The viral vector may, for example, a lentivirus, a retrovirus, an adenovirus, an adeno-associated virus (AAV), a vaccinia virus or a herpes simplex virus. Methods for producing and purifying such vectors are known in the art. The viral vector is preferably replication deficient. The vector may, for instance, be a non-viral vector. The non-viral vector may, for instance, be a DNA plasmid, a naked nucleic acid, a nucleic acid complexed with a delivery vehicle, or an artificial virion. The non-viral vector may be a human artificial chromosome. When the non-viral vector is a nucleic acid complexed with a delivery vehicle, the delivery vehicle may be a liposome, virosome, or immunoliposome. Integration of a plasmid vector may be facilitated by a transposase such as sleeping beauty or PiggyBAC.

### Method of Producing an In Vitro Model of Pyroptosis

The disclosure provides a method of producing an in vitro model of pyroptosis. The method comprises introducing the vector of the disclosure to cells, expressing in the cells the one or more virus-related proteins encoded by the polynucleotide comprised in the vector.

Any of the aspects described above in connection with the vector or the in vitro model of the disclosure may apply to the method of producing an in vitro model of pyroptosis. In particular, the cells to which the vector is introduced may be any of those described above in connection with the in vitro model. The vector may be any vector described above.

Introduction of the vector to the cells modifies the cells, such that they comprise a polynucleotide encoding one or more virus-related proteins that activate the NLRP3 inflammasome. As discussed above, activation of the NLRP3 inflammasome drives pyroptosis. The vector may be introduced to the cells using any method known in the art. For instance the cells may be transfected or transduced with the vector

The term “transduction” may be used to describe virus-mediated nucleic acid transfer. A viral vector may be used to transduce the cell with the polynucleotide encoding one or more virus-related proteins that activate the NLRP3 inflammasome. Conventional viral based expression systems could include retroviral, lentivirus, adenoviral, adeno-associated (AAV) and herpes simplex virus (HSV) vectors for gene transfer. Methods for producing and purifying such vectors are known in the art. Transduction may be in vitro or ex vivo.

The term “transfection” may be used to describe non-virus-mediated nucleic acid transfer. Transfection may be in vitro or ex vivo. Any vector capable of transfecting the cells with the polynucleotide encoding one or more virus-related proteins that activate the NLRP3 inflammasome may be used, such as conventional plasmid DNA or RNA transfection. A human artificial chromosome and/or naked RNA and/or siRNA may be used to transfect the cell with the nucleic acid sequence or nucleic acid construct. DNA plasmids, naked nucleic acid, and nucleic acid complexed with a delivery vehicle, such as a liposome. Methods of non-viral delivery of nucleic acids include lipofection, microinjection, biolistics, virosomes, liposomes, immunoliposomes, polycation or lipid: nucleic acid conjugates, naked DNA, artificial virions, and agent-enhanced uptake of DNA. Vector uptake may be enhanced by several known transfection techniques, for example those including the use of transfection agents. Examples of these agents includes cationic agents, for example, calcium phosphate and DEAE-Dextran and lipofectants, for example, lipofectAmine, fugene and transfectam.

Nanoparticle delivery systems may be used to transfect the cell with the polynucleotide encoding one or more virus-related proteins that activate the NLRP3 inflammasome. Such delivery systems include, but are not limited to, lipid-based systems, liposomes, micelles, microvesicles and exosomes.

Following introduction of the vector, the cells express the one or more virus-related proteins. Methods for determining protein expression are known in the art and described above.

### Method for Determining the Ability of an Agent to Inhibit Pyroptosis

The disclosure provides a method for determining the ability of an agent to inhibit pyroptosis. The method comprises: (a) culturing a first population of cells and a second population of cells for a period of time, wherein (i) the first population and the second population are obtained from the in vitro model of the disclosure, and (ii) the first population is cultured in the presence of the agent and the second population is cultured in the absence of the agent; (b) determining the level of pyroptosis in each of the first and second populations; and (c) using the level determined in (b) to indicate the ability of the agent to inhibit pyroptosis, wherein reduced pyroptosis in the first population relative to the second populations indicates that the agent is capable of inhibiting pyroptosis.

### Populations

The first population and the second population are each obtained from the in vitro model of the disclosure. Preferably, the first population and the second population are each obtained from the same in vitro model of the disclosure. In other words, the first population may be a first aliquot of an in vitro model of the disclosure, and the second population may be a second aliquot of that in vitro model. Thus, the first population and the second population may comprise substantially the same type of cells.

### (a) Culturing

The first and second populations are cultured for a period of time. The first population is cultured separately from the second culture. For instance, the first culture may be cultured in a difference reaction vessel (such as a different flask, or a different well of a plate) from the second population. Methods for cell culture are well-known in the art. The populations of cells may be cultured using any methods known in the art. For example, the cells may be cultured in accordance with the Examples disclosed herein.

The first population is cultured in the presence of the agent. The agent may be present in any concentration. Several cultures may be conducted in parallel, to allow for titration of the agent.

The second population is cultured in the absence of the agent. The absence of the agent is expected to allow pyroptosis to develop and/or progress in the second population. Essentially, the second population provides a negative or “untreated” control for the first population.

Optionally, a third population of cells may be cultured for the same period of time as the first and second population, in the presence of a known anti-pyroptotic drug. The anti-pyroptotic drug may, for instance, be an agent whose administration reduces TSPO signalling in an individual. Such agents are described in detail above. The presence of the drug is expected to prevent or reduce the development and/or progression of pyroptosis in the third population. Essentially, the third population provides a positive control for the first population. The third population is cultured separately from the first and second cultures. The third population is obtained from the in vitro model of the disclosure. Preferably, the third population is obtained from the same in vitro model of the disclosure as the first and second populations. In other words, the third population may a third aliquot of an in vitro model of the disclosure.

The period of time may be of any duration. Preferably, the period time is sufficient to allow pyroptosis to be observed in the second population. Preferably, the period of time is sufficient to allow any anti-pyroptotic effect of the agent to be seen in the first population. Preferably, the period time is sufficient to allow (i) pyroptosis to be observed in the second population and (ii) any anti-pyroptotic effect of the agent to be seen in the second population. For example, the period of time may be around 2 hours to around 96 hours. For instance, the period may be from 2 hour to 12 hours, such as from 4 hours to 10 hours, or from 6 hours to 8 hours, The period may be from 12 hours to 72 hours, such as from 24 hours to 48 hours, or around 36 hours. The period may, for example, be around 16 hours, around 24 hours, around 48 hours, around 72 hours, or longer.

### (b) Determining the Level of Pyroptosis

The level of pyroptosis is determined in each of the first and second populations. When a third population is cultured, the level of pyroptosis is also determined in the third population.

Methods for determining the level of pyroptosis are known in the art. Any known method, or combination of methods, may be used.

For example, pyroptosis can be determined by measuring the level of cell death in each population. Exemplary methods to measure cell death include the use of trypan blue and propidium iodide. For example, cells treated with an agent can be washed once with PBS and then trypsinised. The cells can then be resuspended in 0.5 mL of PBS 1×. Equal volumes of cell suspension can be mixed with Trypan blue and 10 μL of this mix can be applied to a haemocytometer. The number of Trypan blue positive cells can be divided by the total number of cells and then displayed as a percentage of cell death. Propidium iodide can also be used instead. Propidium iodine is a fluorescent membrane impermeant dye that upon inclusion intercalates with double stranded DNA. Cells treated with propidium iodode can be washed with PBS once. The cells can then be incubated for 5 minutes with 2 μg/mL of propidium iodide diluted in PBS and kept in the dark. After incubation media containing propidium iodide can be washed 3 times using PBS. The cells can be left in PBS 1× and imaged using a microscope such as a DMIRB inverted microscope (Nikon) where for example, a minimum of 5 different fields of view can be imaged. From the images, the number of stained cells and total number of cells can be counted. The number of stained (dead) cells were divided by total number of cells and displayed as a percentage cell death.

The level of pyroptosis may be determined by measuring caspase activation. Active caspases are cleaved from their inactive pro-caspase forms during pyroptosis. Caspase cleavage can be detected by western blot, using a specific caspase antibody. Caspase activation assays, which directly measure caspase activation, are also known in the art.

The level of pyroptosis may be determined by measuring gasdermin D cleavage. Pyroptosis involves cleavage of gasdermin D (53 kDa), resulting in a 30 kDa N-terminal fragment. This can be detected by western blot

The level of pyroptosis may be determined by inhibiting or ablating key components of the pyroptotic pathway. Such inhibition or ablation may be used to demonstrate that observed cell death is dependent on pyroptotic molecules, such as caspases. Caspase inhibitors are known in the art and may be used for this purpose.

### (c) Indicating the Ability of the Agent to Inhibit Pyroptosis

The ability of the agent to inhibit pyroptosis is determined using the level of pyroptosis determined in step (b). Specifically, reduced pyroptosis in the first population relative to the second populations indicates that the agent is capable of inhibiting pyroptosis. That is, if the first population has less pyroptosis compared to the second population, the agent is able to inhibit pyroptosis. The ability of the agent to inhibit pyroptosis may correlate with the reduction in pyroptosis determined between the first and second populations. An agent determined to be able to inhibit pyroptosis may be used to prevent or treat a pyroptosis-associated disease, such as any of the pyroptosis-associated diseases described above.

When a third population of cells is cultured as set out above, the drug may represent an accepted or even gold-standard treatment for pyroptosis. Comparing the level of pyroptosis in the first population with the level of pyroptosis in the third population may indicate the effectiveness or efficacy of the agent relative to the drug. For instance, observation of less pyroptosis in the first population than the third population may indicate that the agent is more effective in preventing or reducing pyroptosis than the drug. Observation of more pyroptosis in the first population than the third population may indicate that the agent is less effective in preventing or reducing pyroptosis than the drug.

### EXAMPLES

The following Examples are provided to illustrate the invention, but are not intended to limit the invention.

### Example 1—Materials and Methods

### Cell Culture

BV2 murine microglial cells were maintained in a temperature-controlled, humidified incubator at 37° C. and 5% CO₂ (Hera Cell 240, Thermo Scientific, Essex, UK). TSPO KO microglia were generated using GeneArt™ CRISPR Nuclease Vector with OFP Reporter Kit (Invitrogen, A21174) and subsequently maintained like the Wild Type (WT) BV2.

THP-1 cells (human derived monocytes ATCC® TIB-202), A549 cells (human derived lung epithelial cells ATCC® CCL-185), Vero E6 cells (ATCC® Number CRL-1586™) both obtained from ATCC.

All cell culture procedures were performed using sterile techniques in a Class II safety cabinet. The cells were maintained in Dulbecco's Modified Eagle Medium (DMEM) containing High Glucose (25 mM), L-Glutamine (4 mM) and Sodium Pyruvate (1 mM) (Thermo Fisher, 11995065), which is supplemented with 10% Fetal Bovine Serum (FBS) (Thermo Fisher, 10082147) 1% of 100 U/mL Penicillin and 100 mg/mL streptomycin (Thermo Fisher, 15140122).

### Transfections

Transfections to introduce the TSPO-gene-targeting CRISPR plasmid in BV2 cells were performed using Lipofectamine 3000 transfection reagent (Thermo Fisher, L3000015). The kit was used according to the manufacturer's instructions and optimized for maximum efficiency in BV2 cells. BV2 cells were seeded (2×10⁵) per well in 6 well plates 24 hours prior to transfection. 48 hours post-transfection, the cells were imaged using a DMIRB inverted microscope (Leica, Germany) to assess transfection efficiency, followed by single cell cloning to isolate a homozygous KO.

Lipofectamine 3000 transfection reagent (Thermo Fisher, L3000015) was also used, following optimization, to express the ORF proteins of interest in THP-1 and A549 cells.

### Western Blotting

The cell monolayer was scraped using Greiner cell scrapers (Greiner, 541070) and placed in a 1.5 mL Eppendorf tube in PBS. The cells in the suspension were pelleted by centrifuging at 1200 RPM for 5 min. PBS was discarded, and the pellet was resuspended in cell lysis buffer (50 mM Tris (Sigma, T6066) pH 8.0, 150 mM NaCl, 1% Triton-X (Sigma, T9284)) containing protease inhibitors (Roche, 04693132001) and kept on ice for 20 minutes. The volume was centrifuged at 10 000 RPM for 5 minutes at 4° C. to allow for removal of cellular debris. The supernatants were stored at −20° C. until required. Subcellular fractionation: The 3 subcellular compartments of interest; Cytosol, nucleus, and mitochondria, were separated by differential centrifugation. To isolate mitochondria, sucrose isotonic fractionation buffer (250 mM sucrose, 20 mM HEPES (pH 7.4), 10 mM KCl, 1.5 mM MgCl₂, 1 mM EDTA, 1 mM EGTA) containing protease inhibitor is added to washed confluent plates. The cells were scraped, and the suspension passed through 26 gauge needle with 1 mL syringes 10-12 times before leaving on ice for 20 minutes. The suspension is centrifuged at 3000 RPM for 5 minutes. The subsequent pellet contains the nuclear fraction. The nuclear pellet is washed using fractionation buffer and passed through a 25 gauge needle and 1 mL syringe 10-12 times. This is followed by centrifugation at 3000 RPM for 10 minutes and the nuclear pellet is resuspended in nuclear buffer (standard lysis buffer containing 10% glycerol and 0.1% SDS). The remaining supernatant (after the initial centrifugation) is transferred and centrifuged at 8000 RPM to obtain the mitochondrial pellet. The supernatant, which consists of the cytosolic fraction, is stored at −20° C. until required. The remaining pellet (mitochondrial fraction) is washed using isotonic buffer and centrifuged at 3000 RPM, 5 minutes. Following this the pellet is resuspended in cell lysis buffer and stored −20° C. until required.

DC protein assay-Protein concentrations were quantified using the Detergent compatible (DC) protein assay (Bio Rad, 5000112). 5 μL of the sample is applied in duplicate to 96-well microplates. Alongside this, 9 standards containing BSA diluted in the same buffer as the samples are run in duplicate. The standards ranged from BSA concentrations of 2 mg/ml to 0.025 mg/mL. 25 μL of Reagent A* (consisting of 20 μL of reagent S to each ml of reagent A) was added to all the wells, followed by 200 μL of reagent B. The plate is left on a rocker to gently mix the reagents for 15 minutes. The absorbance was subsequently read using a plate reader (Tecan Infinite M200 Pro, UK) at 695-750 nm. The BSA standard curve was used to determine the protein concentration (μg/μL) of the samples.

Gel electrophoresis and protein transfer—The volume of protein required to achieve 20 μg of sample was aliquoted into Eppendorf's, and corresponding volume of 5× Laemmli buffer (10% SDS, 50% glycerol, 25% 2-mercaptoethanol, 0.02% bromophenol blue, and 0.3125 M Tris HCl, pH approx. 6.8) added to the samples. Samples were boiled at 95° C. for 5 minutes and subsequently spun down before being loaded. Bio-Rad Mini-PROTEAN tetra system electrophoresis unit was used. The gels were run using a running buffer (Tris-Base 1.5 grams, Glycine 7.5 grams, SDS 0.5 grams made up to 500 mL) and PageRuler™ Plus Prestained Protein Ladder (Thermo Fisher, 26619) was used to aid identification of protein band. Voltage settings were as follows: 80 V for 20 minutes, followed by voltage turned up to 120 V for another 1 hour 30 minutes. After protein separation, the proteins were transferred onto a PVDF membrane (activated in methanol) in a transblot cell (Bio-Rad, UK) in ice-cold transfer buffer (Glycine 0.15 M, Trizma Base 0.025 M, 20% Methanol) at 100 V for 1 hour at RT.

Immunoprobing—After transfer the blots were blocked in 5% (w/v) solution of milk powder in TBS-T (50 mM Tris, 150 mM NaCl, 0.05% Tween 20 (Sigma, P9416)) for 1 hour at RT while rolling, preventing non-specific binding of the primary antibody. The membrane was subsequently incubated with the appropriate diluted antibody in milk at 4° C. overnight. Following primary antibody incubation, the membranes were washed 3×5 minutes with 5% (w/v) solution of milk powder in TBS-T at RT. The membrane was incubated for 1 hour with the corresponding peroxidase conjugated secondary antibody diluted in milk. Membranes were then washed 3×5 minutes in TBS-T. To visualize the blot the ECL western blotting detection kit (Amersham, RPN2133) was utilized. Equal amounts of component A and component B were mixed and applied to the membranes. After 5 min incubation the solution is removed. Imaging and visualization was performed using a ChemiDoc™ MP System (Bio-Rad, 1708280) for varying time points until the desired band density for each antibody was achieved.

### Cell Death

To assess cell death between cell lines and treatments staining using propidium iodide was performed. Treated cells were washed once with PBS and then trypsinised. Propidium instead is a fluorescent membrane impermeant dye that upon inclusion intercalates with double stranded DNA. Cells that were washed with PBS once. The cells were then incubated for 5 minutes with 2 μg/mL of propidium iodide diluted in PBS and is kept in the dark. After incubation media containing PI was washed 3 times using PBS. The cells were left in PBS 1× and imaged using a DMIRB inverted microscope (Nikon) where a minimum of 5 different fields of view are imaged. From the images number of stained cells and total number of cells were counted. The number of stained (dead) cells were divided by total number of cells and displayed as a percentage cell death.

[Infection Protocol with 2019-nCOV]

Vero E6 cells (ATCC® Number CRL-1586™) were maintained in Modified Eagle Medium (MEM) supplemented with 10% heat inactivated fetal bovine serum (FBS) at 37° C. in a humidified atmosphere of 5% CO₂. Vero E6 cells were exposed to SARS-CoV-2 isolate (SARS-CoV-2/Human/ITA/PAVIA10734/2020, EVA-G Ref-SKU: 008V-04005-see https://www.european-virus-archive.com/virus/sars-cov-2-isolate-sars-cov-2humanitapavia107342020-clade-g-d614g-s), in medium without FBS for 1 hour at 37° C./5% CO₂ at a multiplicity of infection (MOI) of 0.001. At the end of the adsorption period, cells were washed and incubated at 37° C. in medium with 2% FBS, and where indicated, treated with Etifoxine (final concentrations of 30 uM) at 0 and 48 hours post infection (h.p.i.). Supernatants were harvested at 24, 48 and 72 h.p.i., while cells at 48, and 72 h.p.i. In addition, at each time-point, cytopathic effect (CPE) was evaluated by light microscope, and viable cells were counted using Trypan Blue (Sigma-Aldrich). The experiments involving replication-competent SARS-CoV-2 isolate were performed in a biosafety level 3 (BSL-3) laboratory at National Institute for Infectious Diseases “L. Spallanzani” (INMI), Rome, Italy.

### Example 2—TSPO is Required for Pyroptotic Cell Death in Murine Immune Cells of the Brain (BV-2

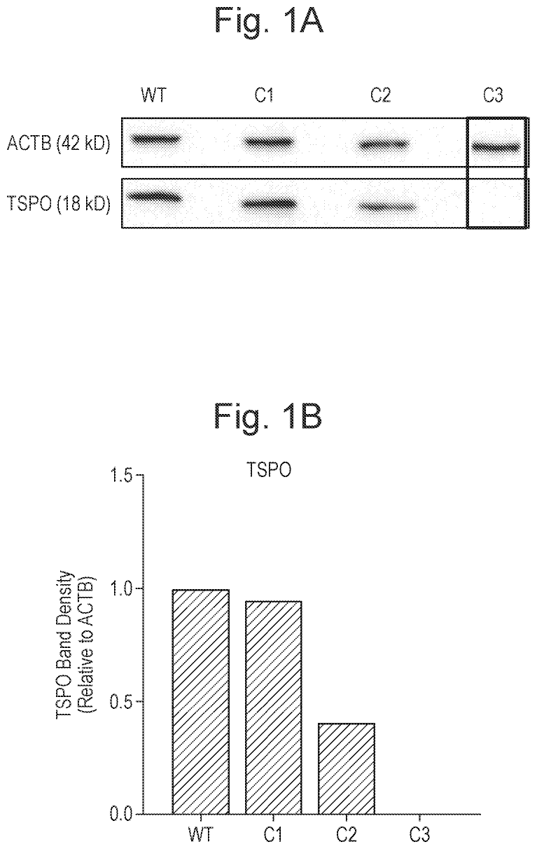

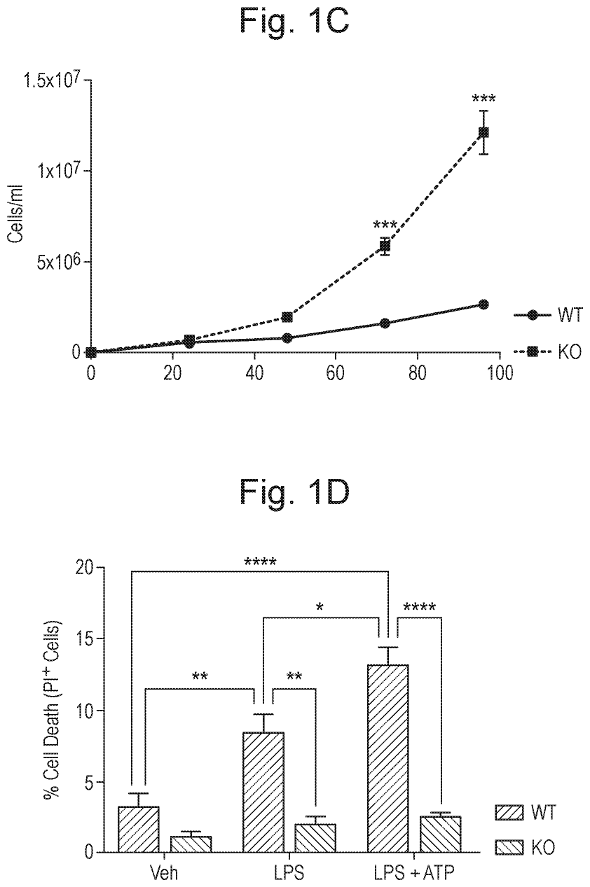

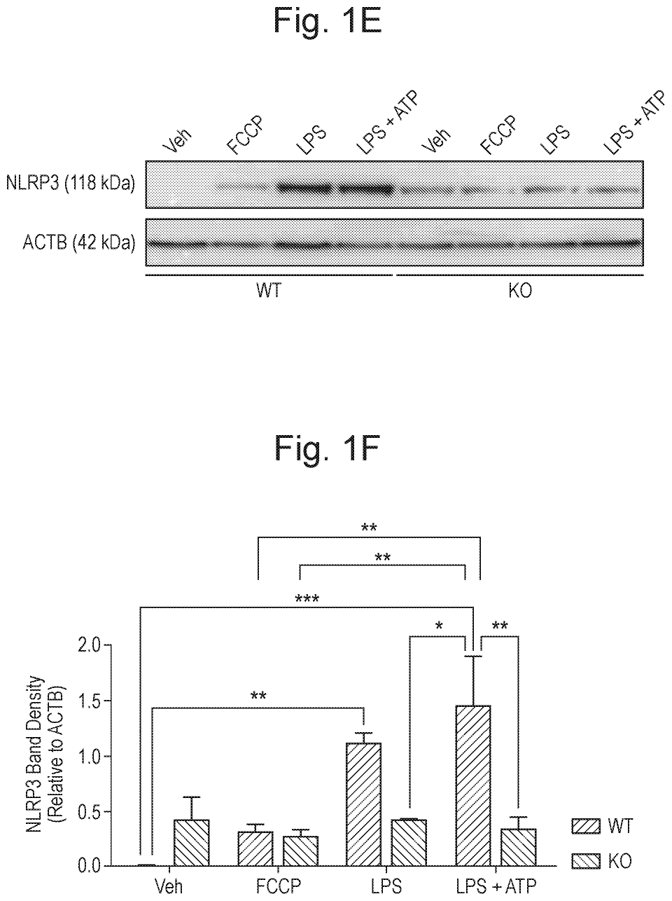

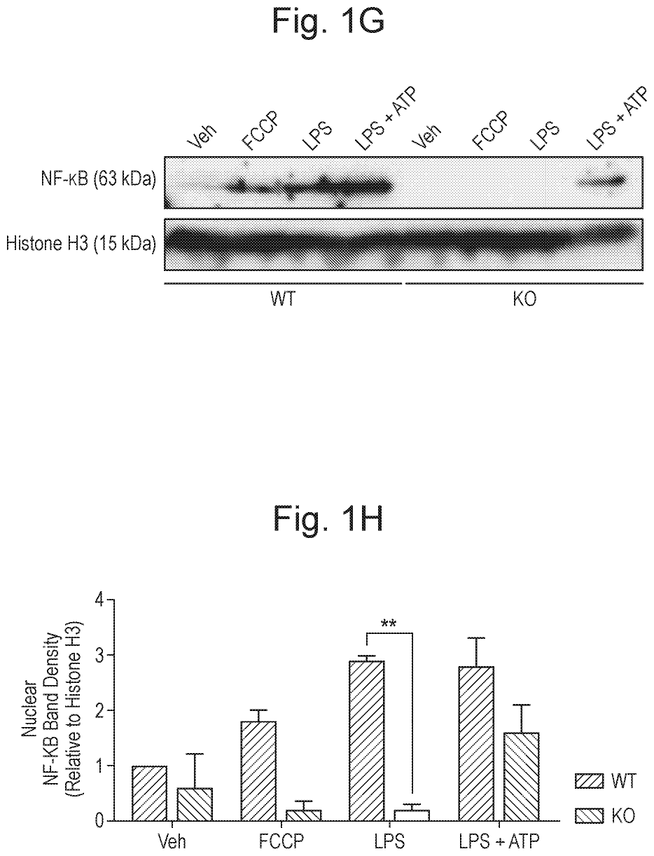

Experiments using brain immune cells (e.g microglia) exposed to endotoxins show that TSPO is linked to the inflammatory type of cell death (pyroptosis) (FIG. 1). The data obtained indicate that ablation of TSPO expression (FIG. 1a, b) protects cells from LPS mediated demise thus allowing proliferation (FIG. 1c, d). This is due to an inhibition of (i) NLRP3 stabilization and (ii) Nf-κB retro-translocation on the nucleus which is required for the expression of pyroptotic proteins (FIG. 1e, f). Protection from LPS induced pyroptosis was also obtained with ligands of the protein (data not shown).

### Example 3—SARS-CoV-2 Proteins ORF8.2b and ORF3a Stabilise TSPO

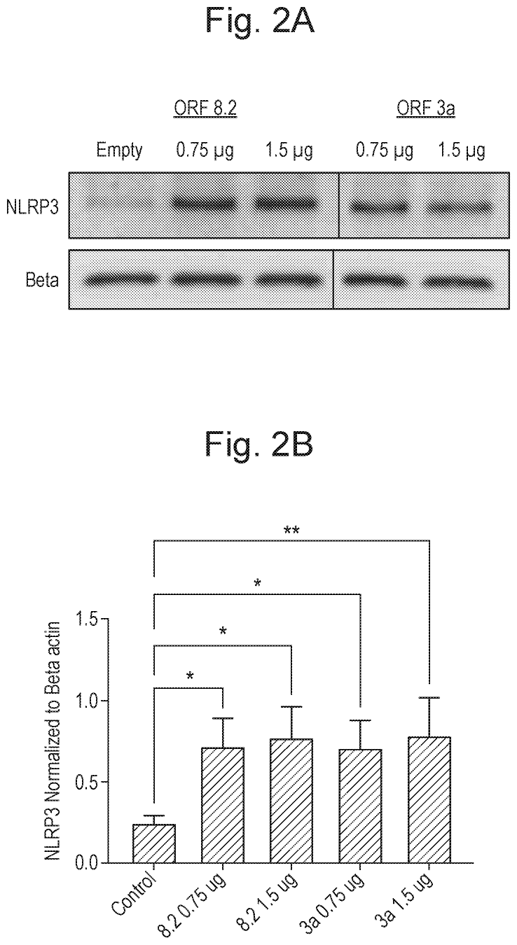

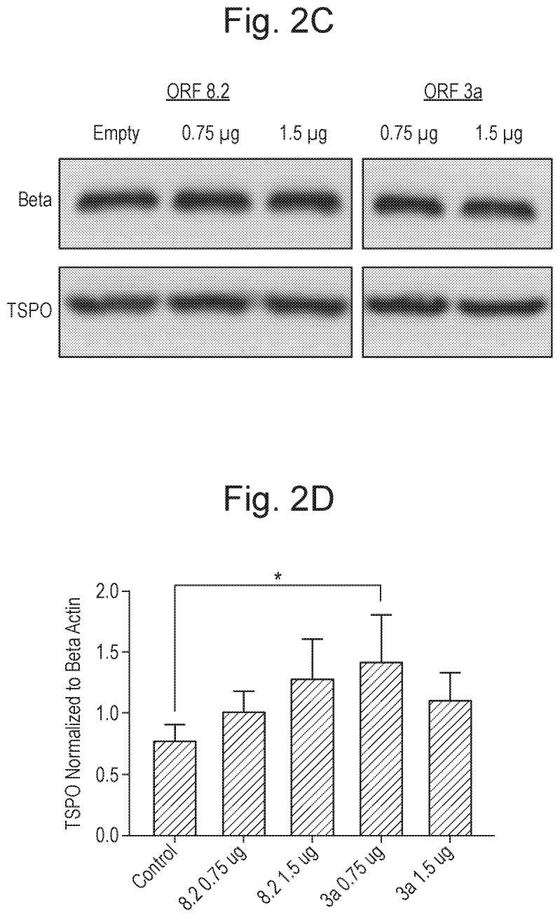

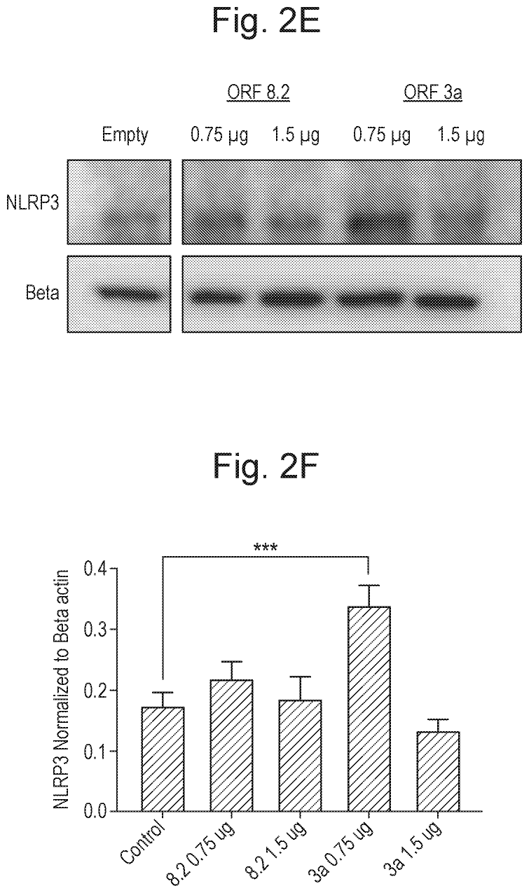

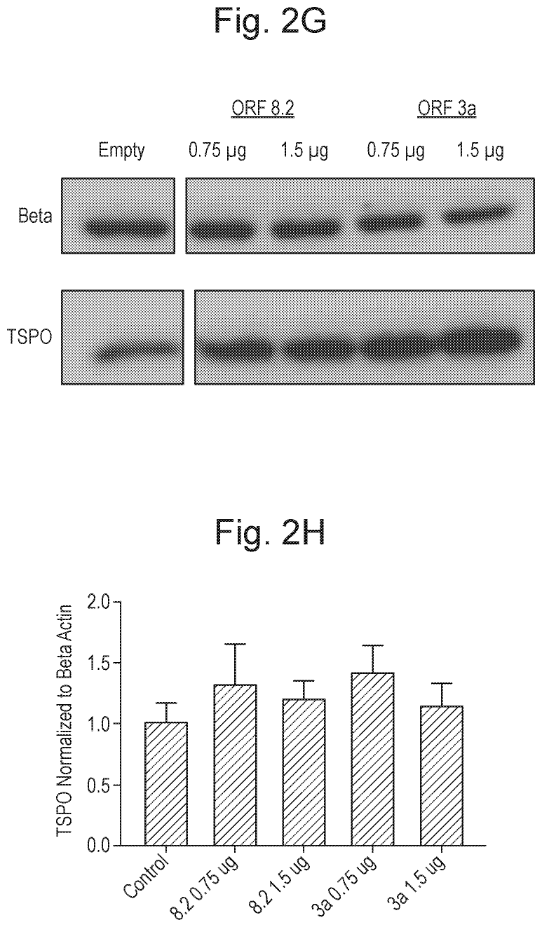

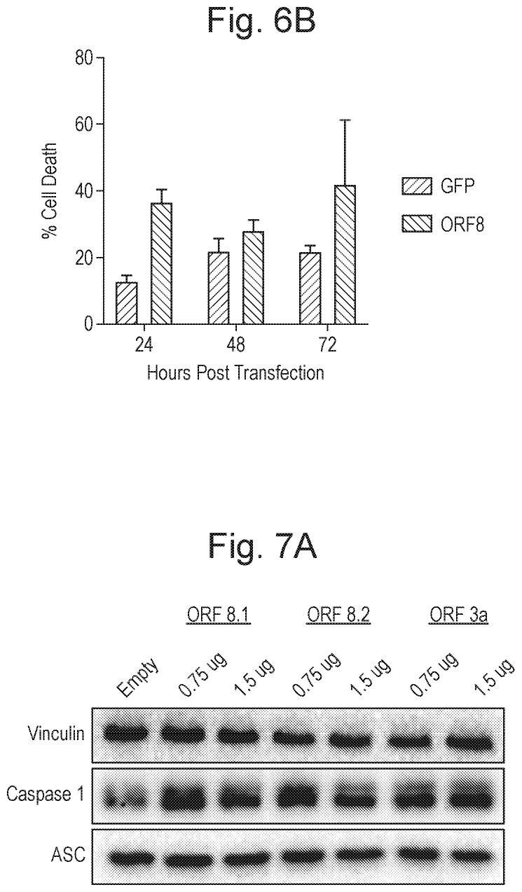

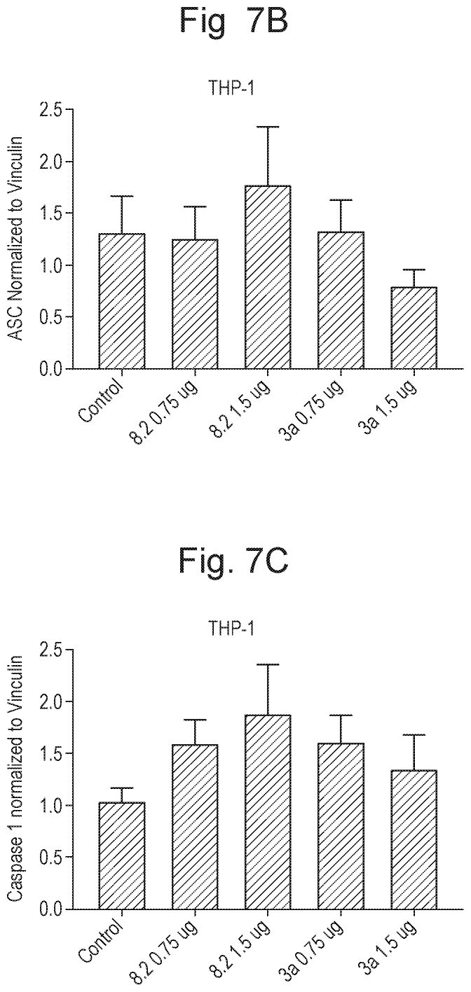

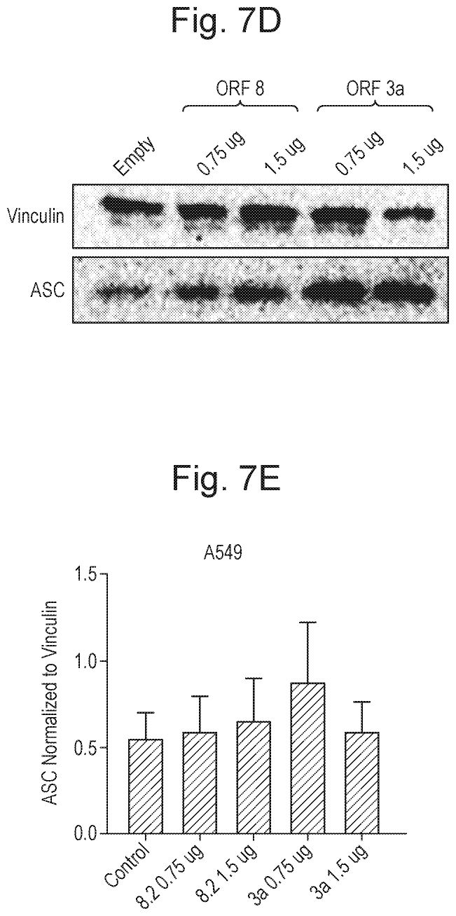

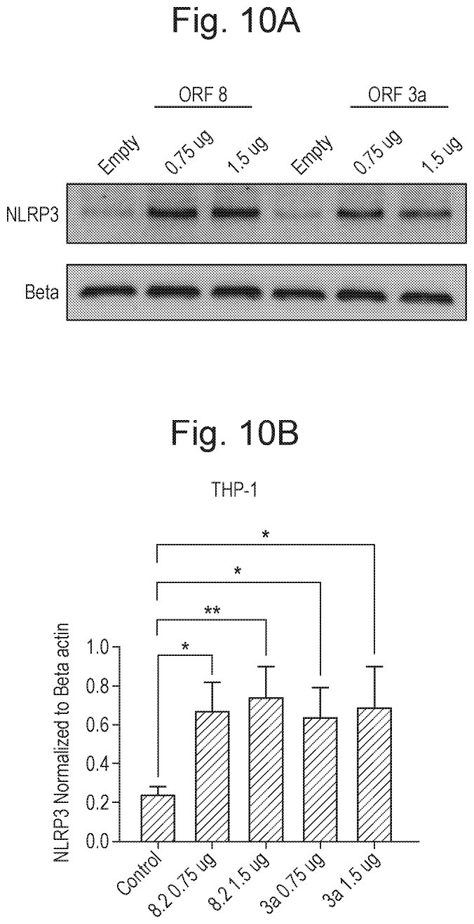

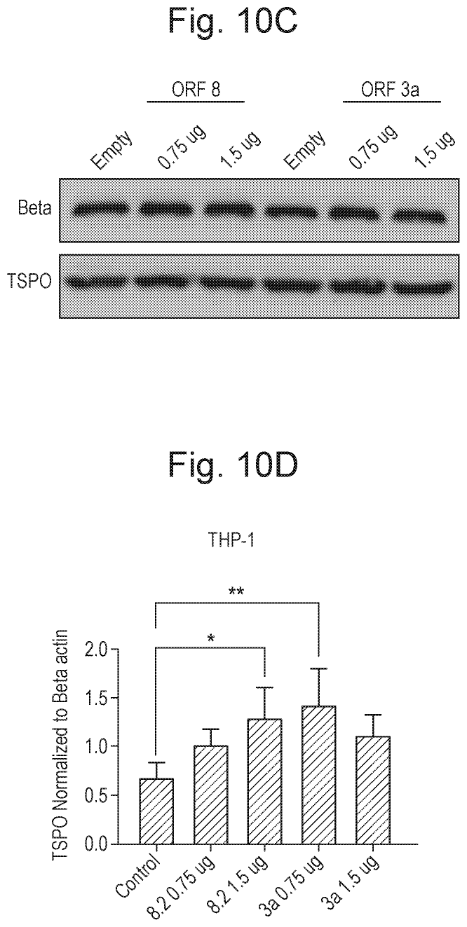

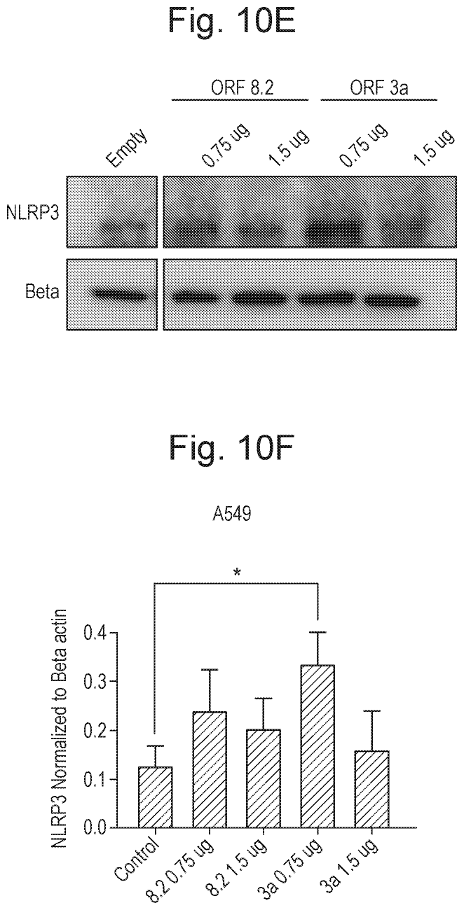

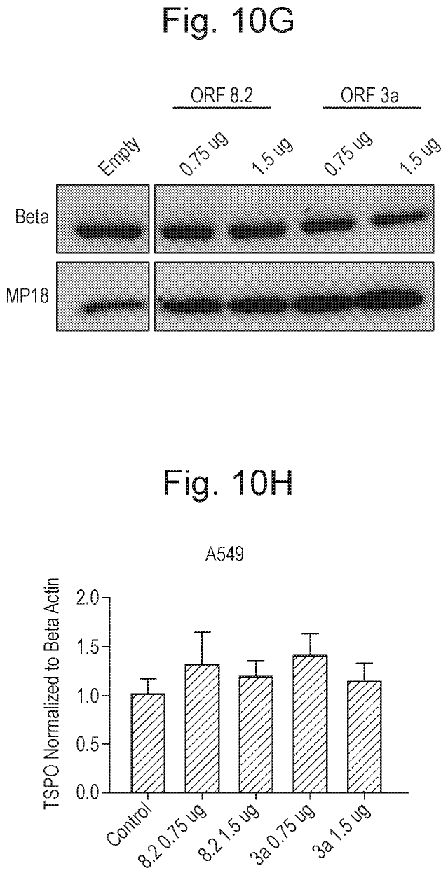

It was hypothesised that TSPO would be overexpressed following 2019-nCOV (SARS-CoV-2) infection, and therefore exploited by pyroptosis to lyse the cells. We devised a model which could mimic in vitro pyroptosis activation by expressing in human monocytes and lung cells the virus-related proteins (ORF 8.2b: Gene Tagged ORF, codon optimized in pCMV6, CAT #: VC102562 from Origene and ORF 3a: Gene Tagged ORF, codon optimized in pCMV6, CAT #: VC102558 from Origene) which trigger NLRP3 inflammasome. Once the involvement of TSPO was established in these models of investigation (FIGS. 2, 6, 7 and 10) we enrolled ligands of the protein to monitor their outcome on cell death.

### Example 4—TSPO Ligands Confer Cytoprotection to Cells Expressing the SARS-CoV-2 Encoded Proteins ORF 8.2b and ORF 3a which Trigger Pyroptosis

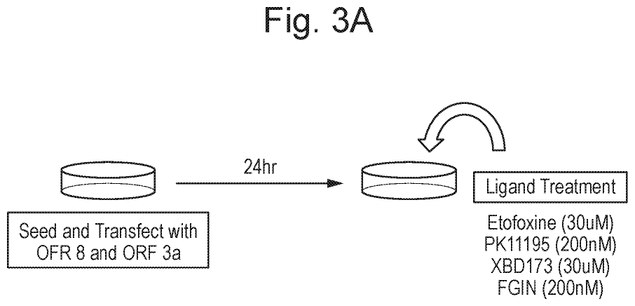

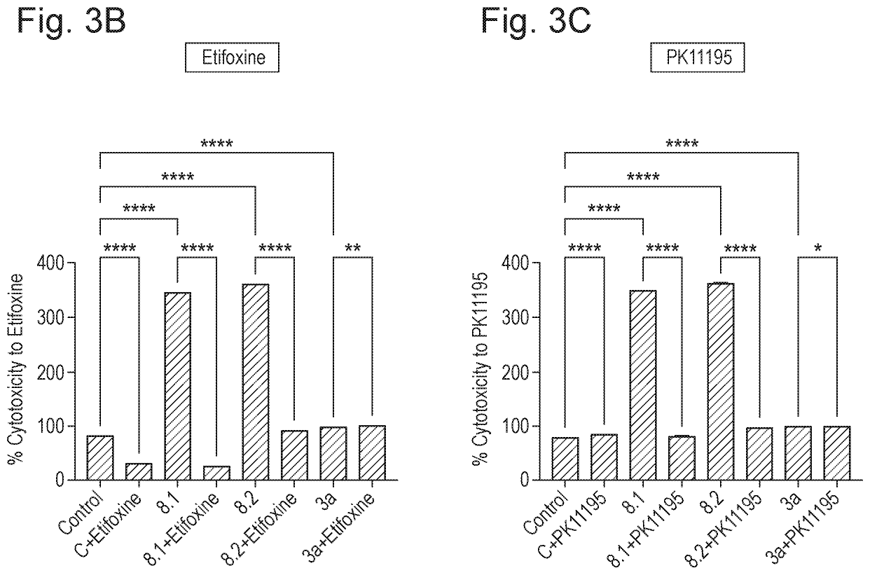

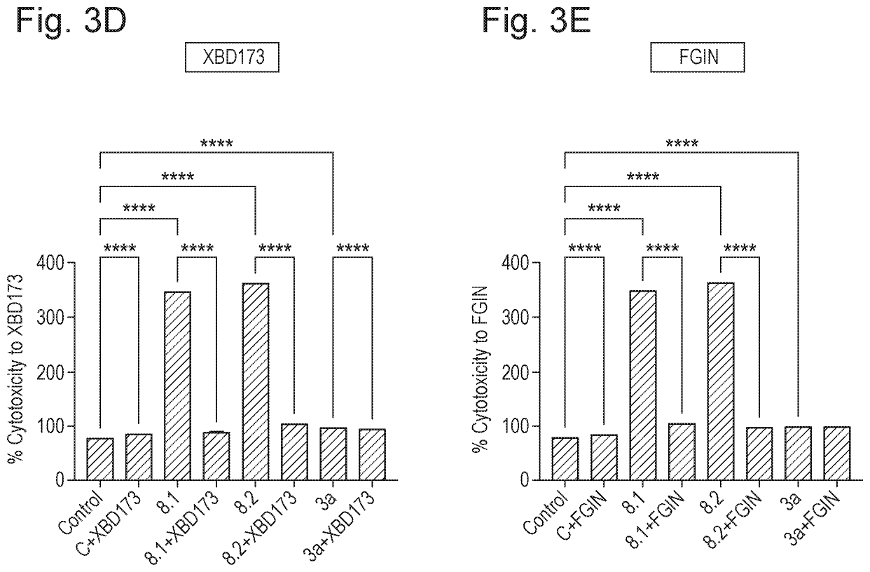

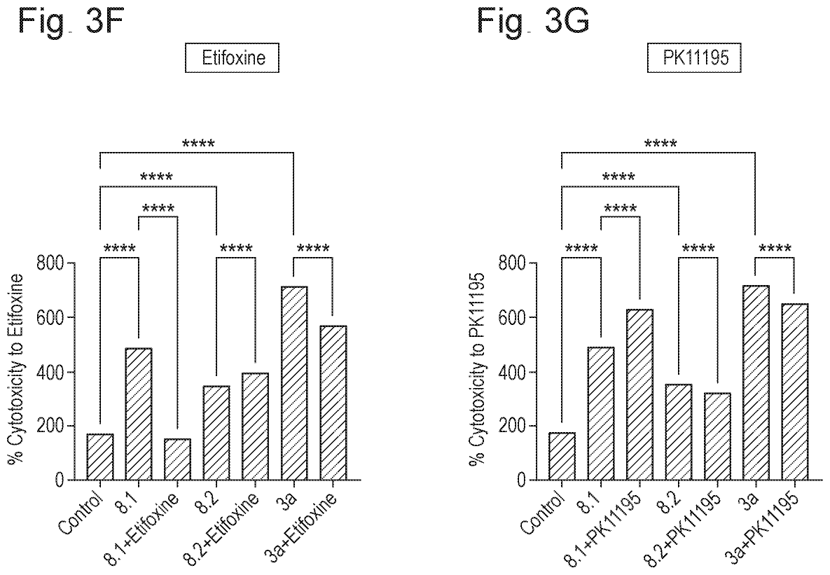

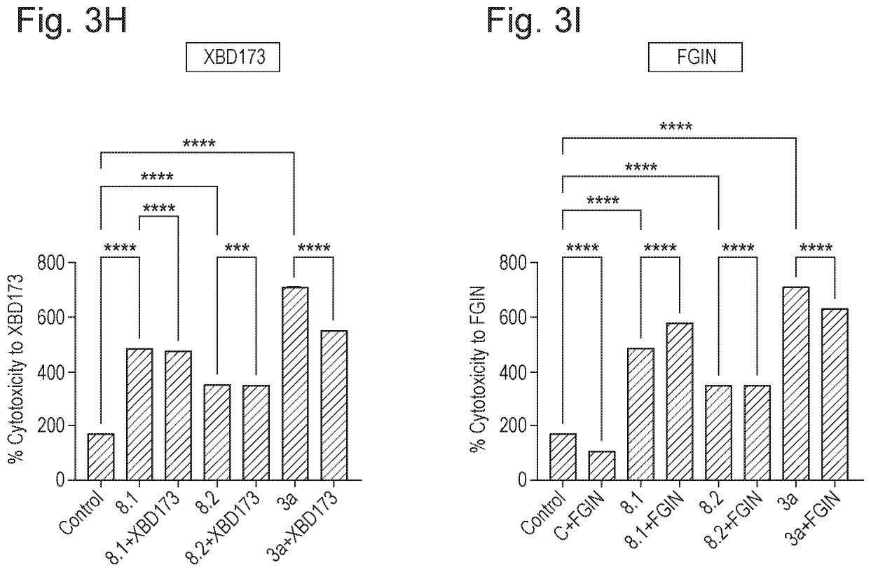

Data in FIG. 3 show the protective effect mediated by compounds in cells in which pyroptosis is triggered by the ORF proteins. This analysis confirmed that TSPO is a target to monitor and counteract pyroptotic damage of cells. The next step was to corroborate both these aspects in cells infected by SARS-CoV-2.

### Example 5—SARS-CoV-2 Upregulates TSPO During Infection

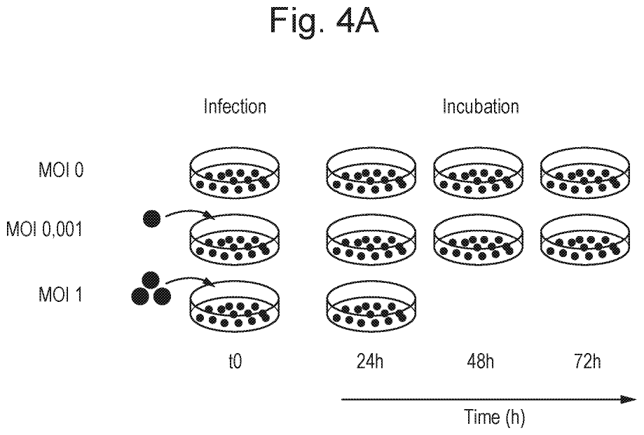

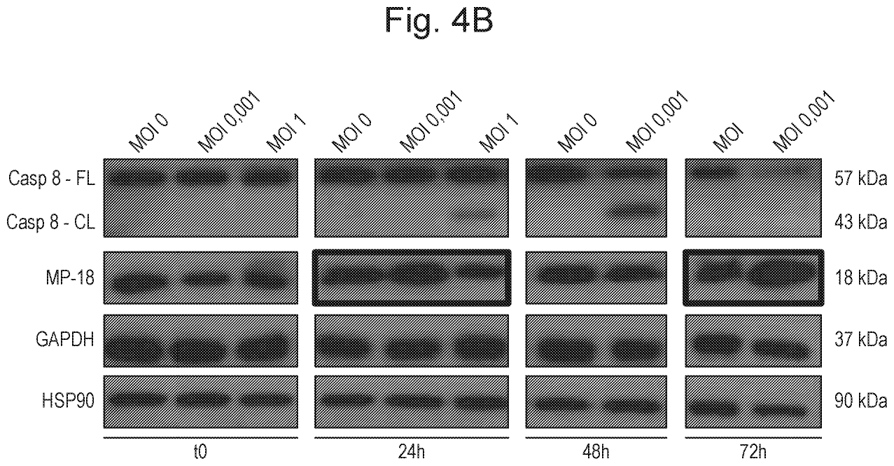

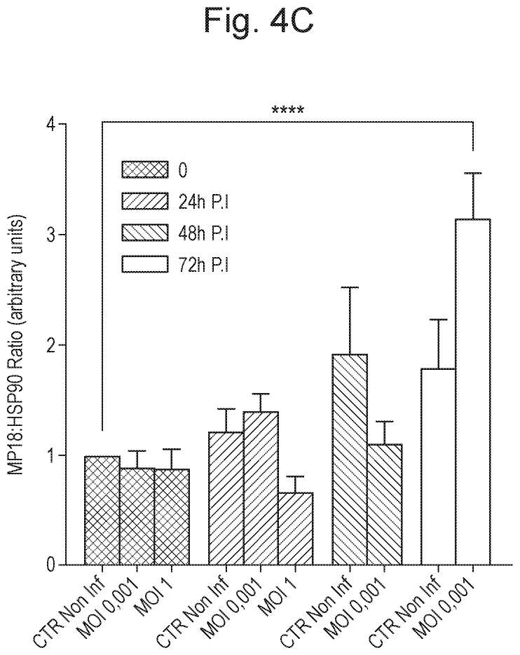

FIG. 4 shows that TSPO is upregulated in a dose dependent manner by the SARS-CoV2.

### Example 6—TSPO Ligands Protect from SARS-CoV-2 Triggered Pyroptosis

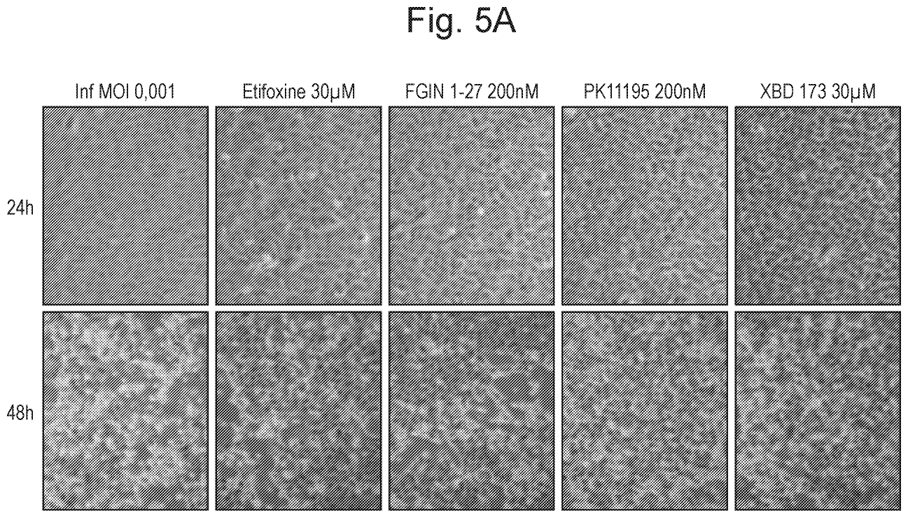

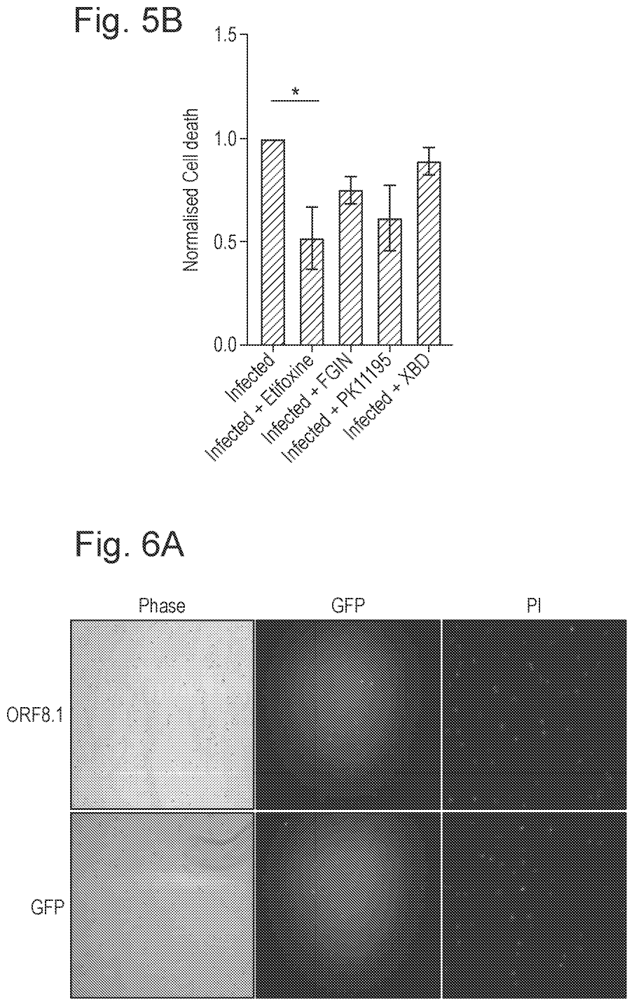

Cellular treatment with TSPO ligands successfully reduces cytotoxicity caused by this strain of coronavirus (FIG. 5). FIG. 5 depicts images of cellular proliferation (Vero cell) 24 hours post-infection using SARS-CoV-2 virions in untreated conditions and then following treatment with MP-18 specific ligands. Among the ligands striking is the protection mediated by Etifoxine (currently commercialised as anxiolytic) which may be used as an anti-pyroptotic medicine to implement the current protocols for the management of COVID-19 patients. Etifoxine reduces cell death by approximately 50% (statistically significant) which is highly indicative of the potential of TSPO ligands as pharmacological inhibitors of COVID-19 associated cell death.

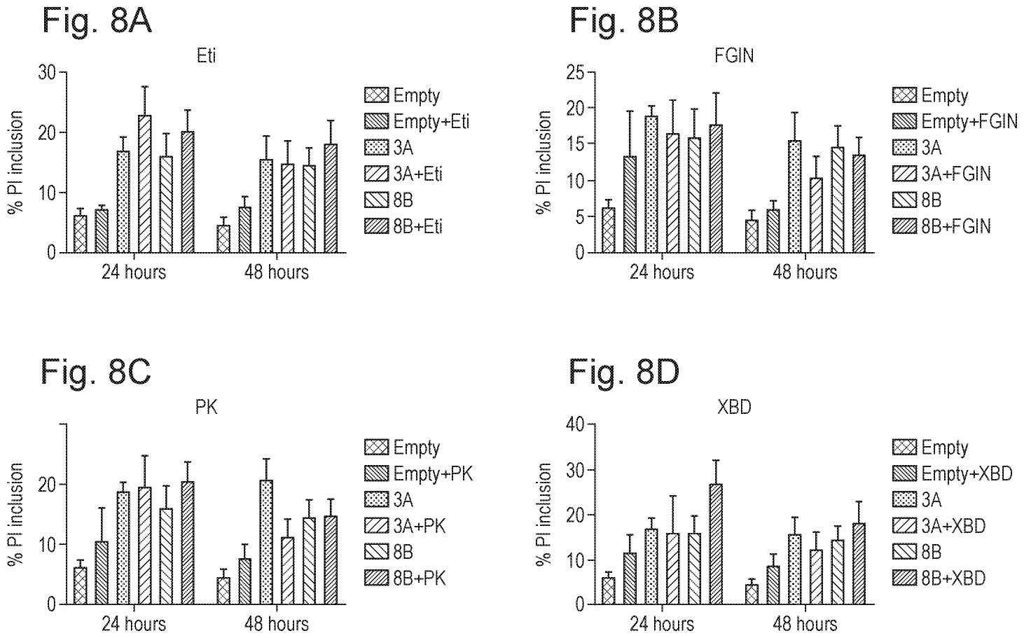

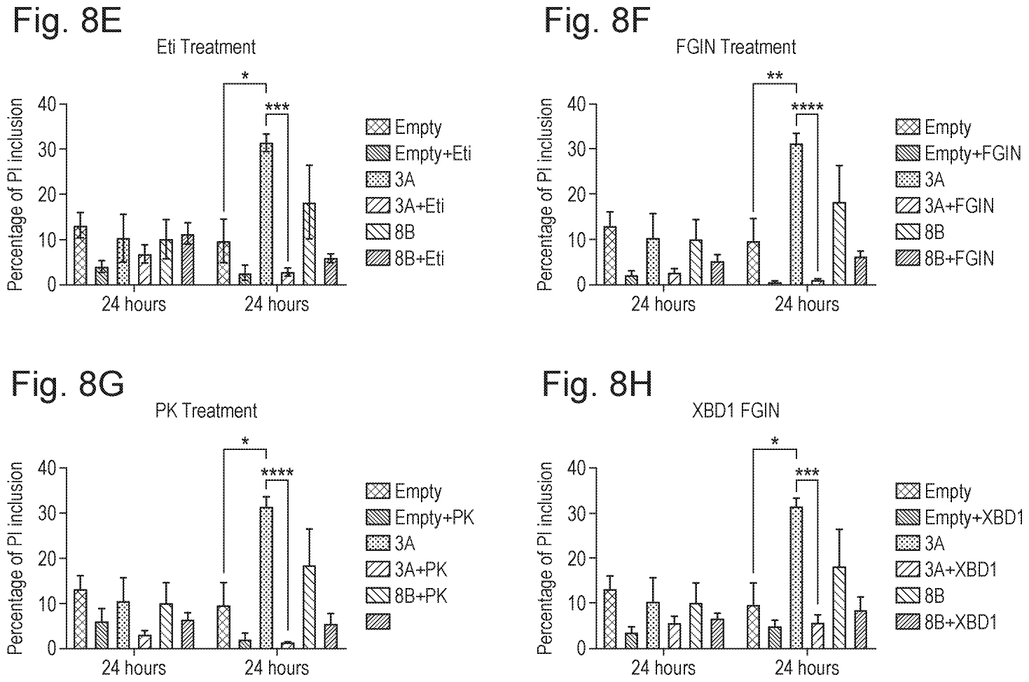

A further experiment was performed in vitro by transfection either A549 or THP-1 cells with 750 ng of SARS-CoV-2 ORF proteins, 3a or 8. At 24 hours following treatment. TSPO ligand (Etifoxine, FGIN, PK11195 or XBD) was administered to transfected cells. PI inclusion was assessed after 24 and 48 hours following transfection and treatment in THP-1 cells (FIG. 8a-d) and A549 (FIG. 8e-h). In THP-1, a decrease was seen after 48 hours treatment with treatment using PK, XBD and FGIN only after initial transfection with ORF 3a. Meanwhile, the results in A549 showed vast differences, with a significant upregulation after transfection with ORF3a and then a statistically significant decrease in ORF3a induced cytotoxicity after treatment with all ligands used.

### Example 7—TSPO Directly Influences Inflammasome Assembly

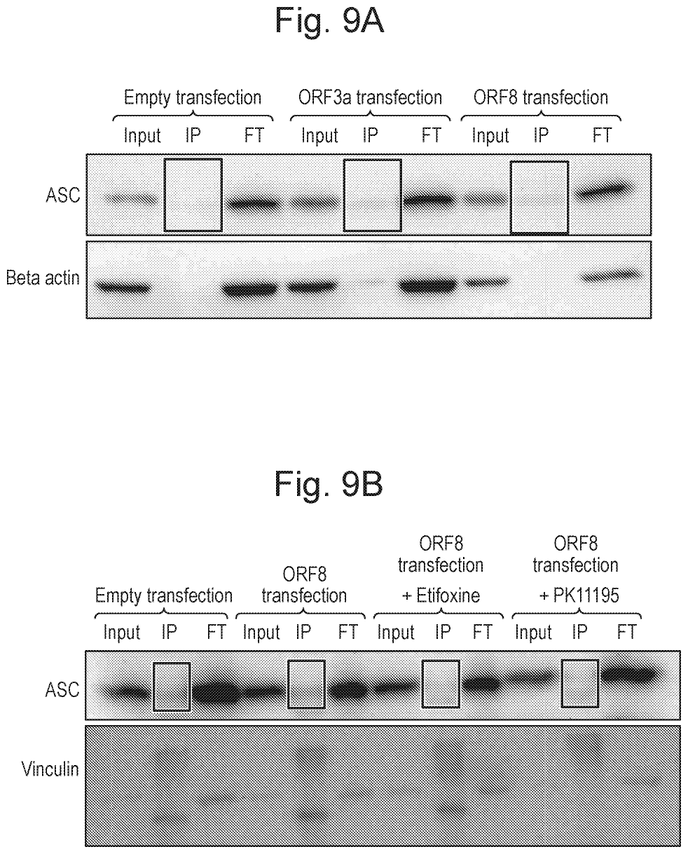

The results set out above indicate that ligands of TSPO have a significant effect on the inflammasome as well as SARS derived ORF proteins. Co-immunoprecipitation studies were undertaken in THP-1 cells to assess whether there is an indirect or direct interaction between TSPO and the proteins that make the NLRP3 inflammasome. Results are shown in FIG. 9.

While the co-immunoprecipitation studies between TSPO-NLRP3 showed no presence of protein when TSPO was used as bait, assessment of ASC showed that there is an increase in ASC levels only after transfection with ORF proteins, as demonstrated in FIG. 9a. This was then followed by a study looking at the transfection of ORF proteins followed by treatment either using 30 uM Etifoxine or 200 nM PK11195. As seen in FIG. 9b there is initially an increase in ASC levels after ORF8 transfection followed by a decrease in protein levels after Etifoxine or PK is given for 24 hours.

The results herein are the first to indicate that regulation of TSPO may directly result in alteration in inflammasome assembly. This would explain why TSPO ligands so robustly disrupt inflammasome mediated cytotoxicity and cell death, as demonstrated above.

1. A method of treating or preventing a disease in an individual, the method comprising reducing pyroptosis by administering to the individual a composition comprising an agent whose administration reduces TSPO signalling in the individual.

2. A composition for use in a method of treating or preventing a disease in an individual, the method comprising reducing pyroptosis by administering the composition to the individual, and the composition comprising an agent whose administration reduces TSPO signalling in the individual.

3. The method of claim 1 or the composition for use of claim 2, wherein the agent comprises a TSPO ligand or a nucleic acid silencing molecule that reduces the expression of TSPO.

4. The method of claim 1 or 3 or the composition for use of claim 2 or 3, wherein the agent reduces activation of the NLR family pyrin domain containing 3 (NLRP3) inflammasome.

5. The method of claim 1, 3 or 4, or the composition for use of claim 2, 3 or 4, wherein the agent reduces TSPO-mediated NRLP3 stabilisation, TSPO-mediated NF-κB nuclear translocation, and/or expression of pyroptotic proteins.

6. The method of any one of claims 1 and 3 to 5, or the composition for use of any one of claims 2 to 5, wherein the composition comprises one or more of Etifoxine, PK11195, XBD173, SSR-180,575 and FGIN.

7. The method or composition for use of any one of claims 3 to 6, wherein the nucleic acid silencing molecule comprises (i) RNA, (ii) DNA, or (iii) RNA and DNA.

8. The method or the composition for use of any one of claims 3 to 7, wherein the nucleic acid silencing molecule comprises or consists of a CRISPR guide RNA, an antisense oligonucleotide (AON), a small interfering RNA (siRNA), a short hairpin RNA (shRNA), or a microRNA (miRNA).

9. The method of any one of claims 1 and 3 to 8, or the composition for use of any one of claims 2 to 8, wherein the disease is characterised by a biochemistry profile indicative of pyroptosis.

10. The method of any one of claims 1 and 3 to 9, or the composition for use of any one of claims 2 to 9, wherein the disease is characterised by increased TSPO expression in a sample obtained from the subject.

11. The method of any one of claims 1 and 3 to 10, or the composition for use of any one of claims 2 to 10, wherein the disease is an infectious disease, optionally wherein the infectious disease is a viral disease, a bacterial disease, a fungal disease or a protozoal disease.

12. The method or composition for use of claim 11, wherein the infectious disease is coronavirus disease 19 (COVID-19), severe acute respiratory syndrome (SARS), or Middle East respiratory disease (MERS).

13. The method of any one of claims 1 and 3 to 10, or the composition for use of any one of claims 2 to 10, wherein the disease is an inflammatory disease, a cancer, a neurodegenerative disease, a cardiovascular disease, a kidney disease, or sepsis.

14. The method of any one of claims 1 and 3 to 13, or the composition for use of any one of claims 2 to 13, wherein the individual is a mammal, optionally wherein the mammal is a human.

15. Use of TSPO expression as a biomarker for pyroptosis.

16. A method of assessing the degree of pyroptosis in an individual, comprising measuring the amount of TSPO expressed in a sample obtained from the individual, wherein the amount of TSPO expressed in the sample correlates with the degree of pyroptosis in the individual.

17. An in vitro model of pyroptosis, comprising cells modified to comprise a polynucleotide encoding one or more virus-related proteins that activate the NLRP3 inflammasome.

18. The in vitro model of claim 17, wherein the virus is SARS-CoV-2, optionally wherein the one or more virus-related proteins comprise ORF 8.2b and/or ORF 3a.

19. The in vitro model of claim 17 or 18, wherein the cells express the one or more virus-related proteins.

20. A vector comprising a polynucleotide as defined in claim 17 or 18.

21. A method of producing an in vitro model of pyroptosis, comprising introducing the vector of claim 20 to cells and expressing the one or more virus-related proteins in the cells.

22. The in vitro model of any one of claims 17 to 19, or the method of claim 21, wherein the cells comprise mammalian cells, optionally wherein the cells comprise human cells.

23. The in vitro model of any one of claims 17 to 19 and 22, or the method of claim 21 or 22, wherein the cells comprise:
(a) monocytes, optionally wherein the monocytes comprise THP-1 cells;
(b) lung cells, optionally wherein the lung cells comprise A549 cells; or
(c) epithelial cells, optionally wherein the epithelial cells comprise Vero cells.

24. A method for determining the ability of an agent to inhibit pyroptosis comprising:
(a) culturing a first population of cells and a second population of cells for a period of time, wherein (i) the first population and the second population are obtained from the in vitro model of any one of claims 17 to 19, 22 and 23, and (ii) the first population is cultured in the presence of the agent and the second population is cultured in the absence of the agent;
(b) determining the level of pyroptosis in each of the first and second populations; and
(c) using the level determined in (b) to indicate the ability of the agent to inhibit pyroptosis, wherein reduced pyroptosis in the first population relative to the second populations indicates that the agent is capable of inhibiting pyroptosis.
